(19) United States
(12) Patent Application Publication
(10) Pub. No.: US 2011/0243957 A1
(43) Pub. Date: Oct. 6, 2011

(54) MATERIALS AND METHODS FOR PREVENTING OR TREATING NEURODEGENERATIVE CONDITIONS ASSOCIATED WITH ABETA PEPTIDE ACCUMULATION

(75) Inventors: Valentina Echeverria Moran, Largo, FL (US); Gary W. Arendash, Prescott, AZ (US)

(73) Assignees: University of South Florida, Tampa, FL (US); United States Department of Veterans Affairs, Washington, DC (US)

(21) Appl. No.: 13/120,633

(22) PCT Filed: Sep. 24, 2009
(86) PCT No.: PCT/US09/58246

§ 371 Date: Jun. 9, 2011

Related U.S. Application Data
(60) Provisional application No. 61/099,761, filed on Sep. 24, 2008.

(51) Int. Cl. A61K 39/395; A61K 31/404; A61K 31/44; A61K 31/166; A61K 31/7088; A61K 38/17; A61P 25/28; C12N 5/0793

(52) U.S. Cl. 424/158.1; 514/418; 514/354; 514/616; 514/44 A; 514/17.7; 435/375

(57) ABSTRACT

The subject invention concerns methods for preventing and/or treating neurodegenerative conditions associated with Abeta peptide accumulation in neural tissue in a human or animal. The subject invention also concerns methods for preventing or treating Alzheimer's disease-like neuropathology in a person or animal having trisomy 21 (Down's syndrome). In one embodiment, a method of the invention comprises administering a therapeutically effective amount of a compound that inhibits function or activity of a Raf protein to a person or animal in need of treatment. In a specific embodiment, the Raf inhibitor is Sorafenib (NEXAVAR). Neurodegenerative conditions contemplated within the scope of the present invention include, for example, Alzheimer's disease and Parkinson's disease. The subject invention also concerns methods for preventing or inhibiting neuronal cell death and/or improving cell viability.

### CROSS-REFERENCE TO RELATED APPLICATION

The present application claims the benefit of U.S. Provisional Application Ser. No. 61/099,761, filed Sep. 24, 2008, which is hereby incorporated by reference herein in its entirety, including any figures, tables, nucleic acid sequences, amino acid sequences, and drawings.

### BACKGROUND OF THE INVENTION

Alzheimer's disease (AD) is the main cause of dementia in the elderly and a progressive degenerative disease of the brain associated with advanced age. AD is characterized by the presence of extracellular amyloid senile plaques in the brain mainly consisting of amyloid-beta peptide (Aβ peptide) (that is generated by proteolytic processing of the trans-membrane protein amyloid precursor protein (APP)) and neurofibrillary tangles composed of aggregated tau protein (a microtubule associated protein). AD pathology is characterized at the neuronal level, by synaptic loss and cell death of selected neuronal populations (Echeverria and Cuello, 2002). There are approximately 17 million people affected by the disease world wild, and it is estimated that by 2050 there will be approximately 25 million affected in the United States. There are no effective therapeutic agents for this disease, new drugs and potential cures are being intensely investigated.

Down's syndrome, also named as chromosome 21 trisomy, is a genetic disorder caused by the presence of an extra 21st chromosome. It is characterized by impairment of cognitive abilities and physical changes and other health concerns such as a higher risk for congenital heart defects. gastroesophageal reflux disease, recurrent ear infections, obstructive sleep apnea, and thyroid dysfunctions. The incidence of Down's syndrome is estimated at 1 per 800 to 1,000 births. The adult patients with Down's syndrome have a much higher incidence of Alzheimer's disease than non-affected individuals. It has been reported that 25% of persons with Down's syndrome develop the disease by age 40, and the rate increases dramatically to 65% after age 60 post-mortem, nearly all adults that suffered from Down's syndrome present Alzheimer's disease pathology including plaques and Aβ accumulation.

### BRIEF SUMMARY OF THE INVENTION

The subject invention concerns methods for preventing and/or treating neurodegenerative conditions associated with Abeta peptide accumulation in neural tissue in a human or animal. In one embodiment, a method of the invention comprises administering a therapeutically effective amount of a compound or composition that inhibits function or activity of a Raf protein to a person or animal in need of treatment. In one embodiment, the Raf-1 inhibitor is GW5074 (shown below), or a pharmaceutically acceptable salt thereof.

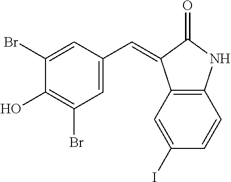

In a specific embodiment, the Raf inhibitor is Sorafenib (NEXAVAR) (shown below), or a pharmaceutically acceptable salt thereof.

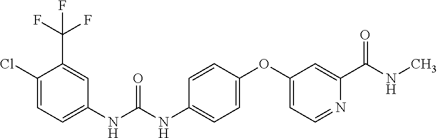

Neurodegenerative conditions contemplated within the scope of the present invention include, for example, Alzheimer's disease and Parkinson's disease.

The subject invention also concerns methods for preventing or treating Alzheimer's disease-like neuropathology in a person or animal having trisomy 21 (Down's syndrome). In one embodiment, a method of the invention comprises administering a therapeutically effective amount of a compound or composition that inhibits function or activity of a Raf protein to a person or animal in need of treatment. In a specific embodiment, the Raf inhibitor is Sorafenib (NEXAVAR).

The subject invention also concerns methods for preventing or inhibiting neuronal cell death and/or improving cell viability. In one embodiment, a method of the invention comprises administering a therapeutically effective amount of a compound or composition that inhibits function or activity of a Raf protein to a person or animal in need of treatment. In a specific embodiment, the Raf inhibitor is Sorafenib (NEXAVAR).

The subject invention also concerns methods for decreasing the synthesis of Abeta peptide in a cell and/or decreasing oligomerization of Abeta peptide. In one embodiment, the method comprises contacting a cell with an effective amount of a compound or composition that inhibits function, activity and/or expression of a Raf protein. In one embodiment, the Raf protein is Raf-1. In one embodiment, the Raf-1 inhibitor is GW5074, or a pharmaceutically acceptable salt thereof. In a specific embodiment, the Raf inhibitor is Sorafenib (NEXAVAR), or a physiologically acceptable salt thereof. In one embodiment, the cell is a cortical cell.

The subject invention also concerns methods for inhibiting the activity and/or decreasing the expression of a Raf protein in a cell. In one embodiment, the method comprises contacting a cell with an effective amount of a compound or composition that inhibits function, activity, and/or expression of a Raf protein. In one embodiment, the Raf protein is Raf-1. In one embodiment, the Raf-1 inhibitor is GW5074, or a pharmaceutically acceptable salt thereof. In a specific embodiment, the Raf inhibitor is Sorafenib (NEXAVAR), or a physiologically acceptable salt thereof. In one embodiment, the cell is a cortical cell.

The subject invention also concerns methods for decreasing or downregulating the expression of an inhibitor of NFκB in a cell. In one embodiment, the method comprises contacting a cell with an effective amount of a compound or composition that inhibits activity, function, and/or expression of a Raf protein. In one embodiment, the NFκB inhibitor is IκB-α. In one embodiment, the Raf protein is Raf-1. In one embodiment, the Raf-1 inhibitor is GW5074, or a pharmaceutically acceptable salt thereof. In a specific embodiment, the Raf inhibitor is Sorafenib (NEXAVAR), or a physiologically acceptable salt thereof. In one embodiment, the cell is a cortical cell.

### BRIEF DESCRIPTION OF THE DRAWINGS

FIGS. 1A-1D show the dysregulation of cRaf-1 in the cortex of APPswe mice. We analyzed cRaf-1 phosphorylation in the cortex of 16-18-month-old APPswe mice and age-matched wildtype control littermates (WT). Cortical tissues were homogenized in lysis buffer containing protease and phosphatase inhibitors, and the immunoreactivity for cRaf-1 phosphorylated at serines 338 and 259 was determined by Western blotting (FIGS. 1D and 1B, respectively), Values were calculated as percentage of wild-type mice levels considered as 100% and expressed as mean=SEM. *Significant difference of the means as analyzed using student t-test.

FIGS. 2A-2C show that Raf-1 inhibitors are neuroprotective against Aβ toxicity. Embryonic rat cortical cells were cultured in Neurobasal/B27 media. After 7 DIV, cells were co-treated with 5 μM Aβ and different concentrations of the Raf inhibitors, GW5074 (FIG. 2A and 2C) and ZM336372 (FIG. 2B). After 48 h, cell viability was analyzed by using MTT (FIGS. 2A and 2B) and calcein/propidium iodide (PI) assays (FIG. 2C). Cell viability values were normalized as percentage of the control cell values considered as 100% and expressed as mean±SEM. ***Significant difference P<0.001. **P<0.01. ns, No significant difference. Aβ, Amyloid beta peptide.

FIGS. 3A-3C show that GW5074 protects cortical cells against Aβ toxicity by inhibiting NFκB. Cortical cells were cultured in Neurobasal/B27 media. After 7 DIV, cells were treated with 5 μM Aβ alone or co-treated with the Raf inhibitor GW5074 and/or the NFκB inhibitor SN50. After 48 h, cell viability was analyzed by using MTT and expressed as percentage of the controls (FIG. 3A). To investigate the effect of GW5074 on NFκB phosphorylation at serine 276, after 7 DIV cortical cells were treated with vehicle (control), 5 μM Aβ and 5 μM Aβ plus 10 μM GW5074 for 48 h. After treatment, cells were homogenized in lysis buffer containing protease and phosphatase inhibitors, and equal amounts of protein samples (60 μg) were analyzed by Western blot (FIG. 3B). The cell viability results were normalized to control values considered as 100% and expressed as mean±SEM. ***Significant difference P<0.001. ns, No significant difference.

FIGS. 4A-4C. Chronic treatment with sorafenib inhibits cRaf-1 and ppERKs in the brain of Tg mice. In Study I, 15-17-month-old Tg mice were treated with sorafenib by gavage (20 mg/kg/day) for 2.0 months. After this time mice were sacrificed and the levels of pcRaf-1[Ser338] and pERK1/2 were analyzed by Western blot as described in Experimental Procedures. Sorafenib-treated Tg mice (n=4) showed significantly lower levels of ppERK (FIG. 4A) and the active form of cRaf-1, pcRaf-1[Ser338] (n=5) (FIG. 4B) in the cortex than untreated Tg mice (n=4). The differences were significant for ppERKs, *P=0.016 and pcRaf-1[Ser338], *P=0.018. (FIG. 4C) Phosphorylation of cRaf-1 at Ser 259 and Ser 338. Examples of the resulting blots are indicated in the upper portion of this figure with lines separating different parts of the same gel. The data are representative of a minimum of two different experiments. NT, wild-type age-matched control littermates.

FIGS. 5A-5E. Sorafenib inhibits NF-κB signaling in the cortex of aged Tg mice. In Study II, 17-19 month-old Tg mice had been treated with sorafenib by gavage (20 mg/kg/day) for four months and their cortical levels of IκBα (Study I), pNF-κB, Cox-2, APP, and iNOS (Study II) protein expression were assessed by Western blot (FIG. 5E). Examples of the resulting blots are indicated in the upper portion of this figure, with lines separating different parts of the same gel. (FIG. 5A) A significant increase (**P=0.002) in the levels of IκB-α was observed in Tg mice treated with sorafenib (n=4) in comparison with control Tg mice (n=3). (FIG. 5B) Sorafenib-treated Tg mice (n=5) showed significantly lower levels of the active form of NF-κB, pNF-κB (Ser276) in cortex compared to control Tg mice (n=6) (**P=0.004; ***P=0.0001). (FIG. 5C) Sorafenib-treated Tg mice (n=4) showed lower levels of Cox-2 in cortex than control Tg mice (n=5). Treatment with sorafenib normalized these levels to the values found in NT mice (n=4) (**P=0.0012; ***P=0.0003). (FIG. 5D) Sorafenib-treated Tg mice (n=5) showed lower levels of iNOS in cortex than untreated Tg mice (n=4) (*P=0.04). These results are mean±SEM, and the data are representative of a minimum of two different experiments. NT, wild type age-matched control littermates.

FIGS. 6A-6C. Sorafenib stimulated the PKA/CREB pathway in the cortex of Tg mice. We analyzed the levels of PKA activity (FIG. 6A) and phospho-CREB (FIG. 6B) in the cortex of 17-19 month-old APPswe (Tg) mice that had been treated with sorafenib or vehicle for four months. Values were normalized against β-tubulin immunoreactivity, which was used as a control for protein loading and transfer. (FIG. 6A) The substantial decrease in cortical PKA activity of control Tg mice vs. NT mice (**P=0.005) was eliminated by sorafenib treatment such that PKA activity was comparable to NT mice. (FIG. 6B) Levels of CREB phosphorylated at Ser 133 in the cortex of control Tg mice (n=4) were reduced in comparison to NT controls (n=3) (*P=0.04), while levels in sorafenib-treated Tg mice (n=4) were significantly higher (**P=0.0075) and no different from NT mice. Examples of p-CREB[Ser 133] blots are shown in FIG. 6C with lines separating different parts of the same gel, along with control β-tubulin blots.

FIGS. 7A-7B. In pre-treatment testing Tg mice showed impairment in RAWM task. Before treatments, Tg and NT mice were analyzed for working memory deficits using a standard RAWM test, as described in the Experimental Procedures section. For the final block of pre-treatment testing, NT and Tg mice performed similarly during the naive Trial 1 (T1). However, Tg mice made significantly more errors (FIG. 7A) and had higher escape latencies (FIG. 7B) during working memory Trial 5 (T5). Nonetheless, both NT and Tg mice were able to improve upon their performance between T1 and T5. *P<0.02 for Tg versus NT group for T5. NT, age-matched wild type littermate mice.

FIGS. 8A-8C. Sorafenib improved working memory in the aged Tg mice. Mice were treated for two months with soratenib and tested using the interference test of working memory. (FIG. 8A) Diagram representing the different steps of the working memory interference test. (FIG. 8B) For the first day of interference testing, both Tg groups were impaired in the three-trial recall (A1-A3). By contrast, sorafenib-treated Tg mice showed much better delayed recall performance (A5) compared to Tg controls (lower). For the last day of interference testing, sorafenib-treated Tg mice had three-trial recall comparable to NT controls and performed substantially better than Tg controls (and identically to NT mice) in proactive interference (FIG. 8C). *P<0.05 or higher level of significance versus NT group; **P<0.01 or higher level of significance versus both other groups.

### DETAILED DESCRIPTION OF THE INVENTION

The subject invention concerns methods for preventing and/or treating neurodegenerative conditions associated with Abeta peptide accumulation and/or aggregation in neural tissue in a human or animal. Neurodegenerative conditions contemplated within the scope of the present invention include, but are not limited to, for example. Alzheimer's disease, and Parkinson's disease. Other neurodegenerative conditions contemplated within the scope of the present invention include, but are not limited to, dementia with Lewy bodies (DLB), dementia pugilistica, Pick's disease, cerebral amyloid angiopathy, and posterior cortical atrophy. In one embodiment, a method of the invention comprises administering a therapeutically effective amount of a compound or composition that inhibits function or activity or expression of a Raf protein (see, for example, GenBank accession No. NM_{—}002880 and NP_{—}002871; Swiss-Prot P04049) to a person or animal in need of treatment. In one embodiment, the Raf protein is Raf-1. Inhibitors of Raf contemplated within the scope of the present invention include, but are not limited to, antibodies and organic molecules that inhibit Raf activity, and nucleic acids, such as antisense, miRNA, and siRNA, that inhibit or interfere with expression of a Raf protein.

In one embodiment, the Raf-1 inhibitor is GW5074 (5-iodo-3-[(3,5-dibromo-4-hydroxyphenyl)methy-lene]-2-indolinone; shown below), or a pharmaceutically acceptable salt thereof.

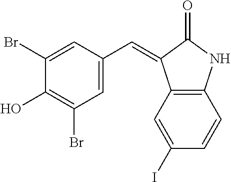

In a specific embodiment, the Raf inhibitor is Sorafenib (NEXAVAR) (shown below; see also published application numbers U.S. 2009/0215835; WO 00/41698; WO 00/042012; WO 06/034796; and WO 06/034797), or a pharmaceutically acceptable salt thereof.

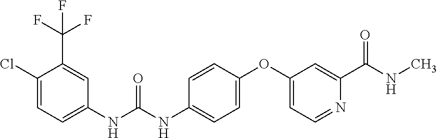

In a more specific embodiment, sorafenib is provided as a tosylate salt.

In another embodiment, the Raf inhibitor is ZM336372 (N-[5-(dimethyl-aminobenzamide)-2-methylphenyl]-4-hydroxybenzamide. Examples of this and other Raf inhibitors contemplated within the scope of the present invention, including antisense and siRNA nucleic acids, are described in Khazak et al. (2007) (e.g., Table 3), the disclosure of which is incorporated by reference herein in its entirety.

Other Raf inhibitors, such as those described in any of U.S. Pat. Nos. 7,307,071, 7,566,716, and 7,491,829 and published international application number WO 2004/064733, are also contemplated for use in the methods of the present invention.

Antibodies, and antigen binding fragments thereof, that bind to and inhibit Raf activity are also contemplated for use in the methods. In one embodiment, the antibody is a human or humanized antibody. In a specific embodiment, the antibody is a monoclonal antibody. Methods and materials for preparing polyclonal and monoclonal antibodies are well known in the art.

The subject invention also concerns methods for preventing or treating Alzheimer's disease-like neuropathology in a person or animal having trisomy 21 (Down's syndrome). In one embodiment, a method of the invention comprises administering a therapeutically effective amount of a compound or composition that inhibits function or activity or expression of a Raf protein to a person or animal in need of treatment. In one embodiment, the Raf protein is Raf-1. In one embodiment, the Raf-1 inhibitor is GW5074 and/or ZM336372, or a pharmaceutically acceptable salt thereof. In a specific embodiment, the Raf inhibitor is Sorafenib (NEXAVAR), or a pharmaceutically acceptable salt thereof.

The subject invention also concerns methods for preventing or inhibiting neuronal cell death and/or improving cell viability. In one embodiment, a method of the invention comprises contacting a cell with an effective amount of a compound or composition that inhibits function or activity or expression of a Raf protein. In one embodiment, the Raf protein is Raf-1. In one embodiment, the Raf-1 inhibitor is GW5074 and/or ZM336372, or a pharmaceutically acceptable salt thereof In a specific embodiment, the Raf inhibitor is Sorafenib (NEXAVAR), or a physiologically acceptable salt thereof. In one embodiment, the cell is a cortical cell.

The subject invention also concerns methods for decreasing the synthesis of Abeta peptide in a cell and/or decreasing oligomerization of Abeta peptide. In one embodiment, the method comprises contacting a cell with an effective amount of a compound or composition that inhibits function, activity and/or expression of a Raf protein. In one embodiment, the Raf protein is Raf-1. In one embodiment, the Raf-1 inhibitor is GW5074 and/or ZM336372, or a pharmaceutically acceptable salt thereof. In a specific embodiment, the Raf inhibitor is Sorafenib (NEXAVAR), or a physiologically acceptable salt thereof. In one embodiment, the cell is a cortical cell.

The subject invention also concerns methods for inhibiting the activity and/or decreasing the expression of a Raf protein in a cell. In one embodiment, the method comprises contacting a cell with an effective amount of a compound or composition that inhibits function, activity, and/or expression of a Raf protein. In one embodiment, the Raf protein is Raf-1. In one embodiment, the Raf-1 inhibitor is GW5074 and/or ZM336372, or a pharmaceutically acceptable salt thereof. In a specific embodiment, the Raf inhibitor is Sorafenib (NEXAVAR), or a physiologically acceptable salt thereof. In one embodiment, the cell is a cortical cell.

The subject invention also concerns methods for decreasing or downregulating the expression of an inhibitor of NFκB in a cell. In one embodiment, the method comprises contacting a cell with an effective amount of a compound or composition that inhibits activity, function, and/or expression of a Raf protein. In one embodiment, the NFκB inhibitor is IκB-α. In one embodiment, the Raf protein is Raf-1. In one embodiment, the Raf-1 inhibitor is GW5074 and/or ZM336372, or a pharmaceutically acceptable salt thereof. In a specific embodiment, the Raf inhibitor is Sorafenib (NEXAVAR), or a physiologically acceptable salt thereof. In one embodiment, the cell is a cortical cell.

An antibody that binds to and inhibits function or activity of a Raf protein, such as Raf-1, can be used in the methods of the present invention. The term “antibody” includes antibody fragments (an antigen binding portion of an antibody), as are known in the art, including Fab or Fab₂, single chain antibodies (Fv for example), chimeric antibodies, etc., either produced by the modification of whole antibodies or those synthesized de novo using recombinant DNA technologies. The term “antigen-binding fragment” or “antigen-binding portion” of an antibody (or simply “antibody portion,” or “fragment”), as used herein, refers to one or more fragments of a full-length antibody that retain the ability to specifically bind to an antigen. Examples of binding fragments encompassed within the term “antigen-binding fragment” of an antibody include (i) a Fab fragment, a monovalent fragment consisting of the VL, VH, CL and CH1 domains; (ii) a F(ab′)2 fragment, a bivalent fragment comprising two Fab fragments linked by a disulfide bridge at the hinge region; (iii) a Fd fragment consisting of the VH and CH1 domains; (iv) a Fv fragment consisting of the VL and VH domains of a single arm of an antibody, (v) a dAb fragment (Ward et al., 1989), which consists of a VII domain; and (vi) an isolated complementarity determining region (CDR). Furthermore, although the two domains of the Fv fragment, VL and VH, are coded for by separate nucleic acids, they can be joined, using recombinant methods, by a synthetic linker that enables them to be made as a single protein chain in which the VL and VH regions pair to form monovalent molecules (known as single chain Fv (scFv); see e.g., Bird et al., 1988; Huston et al., 1988). Such single chain antibodies are also intended to be encompassed within the term “antigen-binding fragment” or “antigen-binding portion” or “fragment” of an antibody. These antibody fragments are obtained using conventional techniques known to those with skill in the art, and the fragments are screened for utility in the same manner as are intact antibodies. The term “monoclonal antibody” or “monoclonal antibody composition”, as used herein, refers to a population of antibody molecules that contain only one species of an antigen binding site capable of immunoreacting with a particular epitope. A monoclonal antibody composition thus typically displays a single binding affinity for a particular protein with which it immunoreacts.

Anti-protein/anti-peptide antisera or monoclonal antibodies can be made as described herein by using standard protocols (See, for example, Harlow and Lane, 1988). For example, a Raf protein, or a portion or fragment thereof, can be used as an immunogen to generate antibodies that bind the component using standard techniques for polyclonal and monoclonal antibody preparation. The full-length component protein can be used or, alternatively, antigenic peptide fragments of the component can be used as immunogens.

Typically, a peptide is used to prepare antibodies by immunizing a suitable subject, (e.g., rabbit, goat, mouse or other mammal) with the immunogen. An appropriate immunogenic preparation can contain, for example, a recombinant Raf protein or peptide or a chemically synthesized protein or peptide. The preparation can further include an adjuvant, such as Freund's complete or incomplete adjuvant, or one or more similar immunostimulatory agents. Immunization of a suitable subject with an immunogenic component or fragment preparation induces a polyclonal antibody response.

Additionally, antibodies produced by genetic engineering methods, such as chimeric and humanized monoclonal antibodies, comprising both human and non-human portions, which can be made using standard recombinant DNA techniques, can be used. Such chimeric and humanized monoclonal antibodies can be produced by genetic engineering using standard DNA techniques known in the art, for example using methods described in U.S. Pat. No. 4,816,567; Better et al., 1988; Liu et al., 1987b; Liu et al., 1987a; Sun et al., 1987; Nishimura et al., 1987; Wood et al., 1985; Shaw et al., 1988; Morrison, 1985; Oi et al., 1986; U.S. Pat. No. 5,225,539; Jones et al., 1986; Verhoeyan et al., 1988; and Beidler et al., 1988.

In addition, a human monoclonal antibody directed against Raf proteins can be made using standard techniques. For example, human monoclonal antibodies can be generated in transgenic mice or in immune deficient mice engrafted with antibody-producing human cells. Methods of generating such mice are described, for example, in Wood et al. PCT publication WO 91/00906, Kucherlapati et al. PCT publication WO 91/10741; Lonberg et al. PCT publication WO 92/03918; Kay et al. PCT publication WO 92/03917; Kay et al. PCT publication WO 93/12227; Kay et al. PCT publication 94/25585; Rajewsky et al. PCT publication WO 94/04667; Ditullio et al. PCT publication WO 95/17085; Lonberg et al., 1994; Green et at., 1994; Morrison et al., 1994; Bruggeman et al., 1993; Choi et al., 1993; Tuaillon et al., 1993; Bruggeman et al., 1991; Duchosal et al. PCT publication WO 93/05796; U.S. Pat. No. 5,411,749; McCune et al., 1988, Kamel-Reid et al., 1988; Spanopoulou, 1994; and Shinkai et al., 1992. A human antibody-transgenic mouse or an immune deficient mouse engrafted with human antibody-producing cells or tissue can be immunized with Raf proteins or an antigenic peptide thereof, and splenocytes from these immunized mice can then be used to create hybridomas. Methods of hybridoma production are well known.

Human monoclonal antibodies can also be prepared by constructing a combinatorial immunoglobulin library, such as a Fab phage display library or a scFv phage display library, using immunoglobulin light chain and heavy chain cDNAs prepared from mRNA derived from lymphocytes of a subject (see, e.g., McCafferty et al. PCT publication WO 92/01047; Marks et al., 1991; and Griffiths et al. 1993). In addition, a combinatorial library of antibody variable regions can be generated by mutating a known human antibody. For example, a variable region of a human antibody known to bind a Raf protein can be mutated by, for example, using randomly altered mutagenized oligonucleotides, to generate a library of mutated variable regions which can then be screened to bind to Raf proteins. Methods of inducing random mutagenesis within the CDR regions of immunoglobin heavy and/or light chains, methods of crossing randomized heavy and light chains to form pairings and screening methods can be found in, for example, Barbas et al. PCT publication WO 96/07754; Barbas et al., 1992.

Expression of one or more target genes can be inhibited or down-regulated using standard methods known in the art. In one embodiment, expression of one or more Raf genes is suppressed or down-regulated. In a specific embodiment, expression of the Raf-1 gene is suppressed or down-regulated. In one embodiment, expression of a target gene is down-regulated using antisense technology. In still another embodiment, expression of a target gene is down-regulated using RNA interference (RNAi) technology, including, for example, the use of short interfering RNA (siRNA). Expression and/or activity (e.g., enzymatic activity) of a protein encoded by a target gene can also be inhibited.

Antisense technology can be used to inhibit expression of a target Raf gene. In antisense methodologies, a nucleic acid that hybridizes with a nucleotide sequence of an mRNA of a target gene is provided. The antisense nucleic acid can hybridize to an entire coding strand of a target sequence, or to a portion thereof, or to a non-coding portion of a target sequence or to both a coding and non-coding portion of a target sequence.

Antisense constructs can have, for example, at least about 70%, at least about 75%, at least about 80%, at least about 85%, at least about 90%, at least about 95%, at least about 96%, 97%, 98%, or 99% sequence identity, or up to 100% sequence identity to the portion of the mRNA that the antisense nucleic acid hybridizes with. Antisense nucleic acids can comprise any suitable number of nucleotides. For example, an antisense nucleic acid construct of the invention can comprise at least 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40 or more nucleotides. In one embodiment, the antisense nucleic acid comprises at least about 40, or at least about 50, or at least about 60, or at least about 70, or at least about 80, or at least about 90, or at least about 100, or at least about 150, or at least about 200, or at least about 250, or at least about 300, or at least about 350, or at least about 400, or at least about 450, or at least about 500, or at least about 550, or at least about 600 or more nucleotides.

RNA interference (RNAi) technologies can also be used to inhibit expression of a target Raf gene. In RNAi, a double-stranded RNA molecule that is complementary to all or a portion of an expressed RNA of a target gene is provided in a cell. The double-stranded RNA molecule is processed into smaller RNA molecules which are then processed into a silencing complex which results in inhibition of expression of the target gene, such as by cleavage of target gene mRNA. Generally, the RNAi molecule has 100 or more nucleotides, and more typically has 200 or more nucleotides. RNAi molecules can be provided by introduction and expression in a cell of a nucleic acid construct that results in transcription and production of the RNAi molecule. In one embodiment, RNA interference via expression of a nucleic acid that provides for micro RNA (miRNA) is contemplated within the scope of the invention. miRNAs are generally 19 to 23 nucleotide RNAs that have been processed from a longer precursor RNA comprising hairpin structures. In another embodiment, RNA interference via expression of a nucleic acid that provides for short interfering RNA (siRNA) is contemplated with the scope of the invention. siRNAs are generally 20 to 25 nucleotide RNAs having 3′ overhangs and that have been processed from a longer precursor double-stranded RNA. Methods and materials for RNA interference have been described, for example, in U.S. Pat. Nos. 7,056,704; 7,078,196; 7,365,058; 7,232,086; 6,506,559; 7,282,564; and 7,538,095.

Aptamers are molecules that bind to a specific target molecule. Aptamers can be composed of nucleic acid (e.g., DNA or RNA) or they can be peptides or polypeptides. Methods for preparing aptamers to a target molecule are known in the art and have been described, for example, in U.S. Pat. Nos. 5,475,096; 5,270,163; 5,707,796; 5,763,177; 6,011,577; 5,580,737; 5,567,588; and 5,840,867. Aptamers contemplated within the scope of the present invention include those that bind to a Raf protein or a gene or polynucleotide encoding a Raf protein, or to a polynucleotide or polypeptide that upregulates or promotes expression of a Raf gene or protein.

The subject invention also concerns kits comprising in one or more containers: a compound that inhibits function or activity or expression of a Raf protein, such as Raf-1, or a composition comprising the compound, or a pharmaceutically acceptable salt and/or analog thereof, and optionally one or more compounds used to treat a neurodegenerative disorder. In one embodiment, a kit comprises one or more of sorafenib and/or GW5074 and/or ZM336372 and/or IκB-α, or an isomer or analog thereof. In one embodiment, a kit comprises an antibody and/or aptamer that binds to or inhibits a Raf protein. In another embodiment, a kit comprises an antisense nucleic acid and/or an interfering RNA and/or a siRNA and/or an miRNA that inhibits or interferes with expression of a Raf protein. Kits of the invention can optionally include pharmaceutically acceptable carriers and/or diluents. In one embodiment, a kit of the invention includes one or more other components, adjuncts, or adjuvants as described herein. In one embodiment, a kit of the invention includes instructions and/or packaging materials that describe how to administer and/or how to use a compound or composition of the kit for the treatment of a neurodegenerative disorder. Containers of the kit can be of any suitable material, e.g., glass, plastic, metal, etc., and of any suitable size, shape, or configuration. In one embodiment, a compound of the invention is provided in the kit as a solid, such as a tablet, pill, chewing gum, or powder form. In another embodiment, a compound of the invention is provided in the kit as a liquid or solution. In one embodiment, the kit comprises an ampoule or syringe containing a compound of the invention in liquid or solution form.

We have been investigating therapeutic strategies to reduce neuronal loss and to restore or ameliorate cognitive functions in AD. We have shown that cRaf-1 is up regulated in AD brains and that Raf kinase inhibitors protect cortical neurons against Aβ toxicity in vitro. The treatment of aged APPswe mice, for two months with the FDA approved Raf inhibitor, Sorafenib (Bay-43-900), restored their otherwise impaired working memory. The neurochemical analysis of brain tissues from APPswe mice and wild type age matched control mice treated and untreated with Sorafenib showed that APswe mice presented a decrease in the levels of the inhibitor of NFκB, IκB-α, and the downstream factors, APP and COX-2. The inhibition of NFκB can decrease the synthesis of Aβ and its toxicity by controlling the levels of APP, β-secretase, and inflammatory factors such as COX-2. Sorafenib improves memory and protects cortical cells against Aβ toxicity by decreasing neuroinflammation in the brain.

To provide for the administration of dosages for the desired therapeutic treatment, in some embodiments, pharmaceutical compositions of the invention can comprise between about 0.1% and 45%, and especially, 1 and 15%, by weight of the total of one or more of the compounds based on the weight of the total composition including carrier or diluents. Illustratively, dosage levels of the administered active ingredients can be: intravenous, 0.01 to about 20 mg/kg; intraperitoneal, 0.01 to about 100 mg/kg; subcutaneous, 0.01 to about 100 mg/kg; intramuscular, 0.01 to about 100 mg/kg; orally 0.01 to about 200 mg/kg, and preferably about 1 to 100 mg/kg; intranasal instillation, 0.01 to about 20 mg/kg; and aerosol, 0.01 to about 20 mg/kg of animal (body) weight.

The compounds of the present invention include all hydrates and salts that can be prepared by those of skill in the art. Under conditions where the compounds of the present invention are sufficiently basic or acidic to form stable nontoxic acid or base salts, administration of the compounds as salts may be appropriate. Examples of pharmaceutically acceptable salts are organic acid addition salts formed with acids that form a physiological acceptable anion, for example, tosylate, methanesulfonate, acetate, citrate, malonate, tartarate, succinate, benzoate, ascorbate, alpha-ketoglutarate, and alpha-glycerophosphate. Suitable inorganic salts may also be formed, including hydrochloride, sulfate, nitrate, bicarbonate, and carbonate salts.

It will be appreciated by those skilled in the art that certain of the compounds of the invention may contain one or more asymmetrically substituted carbon atoms which can give rise to stereoisomers. It is understood that the invention extends to all such stereoisomers, including enantiomers, and diastereoisomers and mixtures, including racemic mixtures thereof.

In vivo application of the subject compounds, and compositions containing them, can be accomplished by any suitable method and technique presently or prospectively known to those skilled in the art. The subject compounds can be formulated in a physiologically- or pharmaceutically-acceptable form and administered by any suitable route known in the art including, for example, oral, nasal, rectal, and parenteral routes of administration. As used herein, the term parenteral includes subcutaneous, intradermal, intravenous, intramuscular, intraperitoneal, and intrasternal administration, such as by injection. Administration of the subject compounds of the invention can be a single administration, or at continuous or distinct intervals as can be readily determined by a person skilled in the art.

The compounds of the subject invention, and compositions comprising them, can also be administered utilizing liposome technology, slow release capsules, implantable pumps, and biodegradable containers. These delivery methods can, advantageously, provide a uniform dosage over an extended period of time. The compounds of the invention can also be administered in their salt derivative forms or crystalline forms.

Compounds of the subject invention can be formulated according to known methods for preparing physiologically acceptable compositions. Formulations are described in detail in a number of sources which are well known and readily available to those skilled in the art. For example, Remington's Pharmaceutical Science by E. W. Martin describes formulations which can be used in connection with the subject invention. In general, the compositions of the subject invention will be formulated such that an effective amount of the compound is combined with a suitable carrier in order to facilitate effective administration of the composition. The compositions used in the present methods can also be in a variety of forms. These include, for example, solid, semi-solid, and liquid dosage forms, such as tablets, pills, powders, liquid solutions or suspension, suppositories, injectable and infusible solutions, and sprays. The preferred form depends on the intended mode of administration and therapeutic application. The compositions also preferably include conventional physiologically-acceptable carriers and diluents which are known to those skilled in the art. Examples of carriers or diluents for use with the subject compounds include ethanol, dimethyl sulfoxide, glycerol, alumina, starch, saline, and equivalent carriers and diluents. To provide for the administration of such dosages for the desired therapeutic treatment, compositions of the invention will advantageously comprise between about 0.1% and 99%, and especially, 1 and 15% by weight of the total of one or more of the subject compounds based on the weight of the total composition including carrier or diluent.

Compounds of the invention, and compositions thereof, may be systemically administered, such as intravenously or orally, optionally in combination with a pharmaceutically acceptable carrier such as an inert diluent, or an assimilable edible carrier for oral delivery. They may be enclosed in hard or soft shell gelatin capsules, may be compressed into tablets, or may be incorporated directly with the food of the patient's diet. For oral therapeutic administration, the active compound may be combined with one or more excipients and used in the form of ingestible tablets, buccal tablets, troches, capsules, elixirs, suspensions, syrups, wafers, aerosol sprays, and the like.

The tablets, troches, pills, capsules, and the like may also contain the following: binders such as gum tragacanth, acacia, corn starch or gelatin; excipients such as dicalcium phosphate; a disintegrating agent such as corn starch, potato starch, alginic acid and the like; a lubricant such as magnesium stearate; and a sweetening agent such as sucrose, fructose, lactose or aspartame or a flavoring agent such as peppermint, oil of wintergreen, or cherry flavoring may be added. When the unit dosage form is a capsule, it may contain, in addition to materials of the above type, a liquid carrier, such as a vegetable oil or a polyethylene glycol. Various other materials may be present as coatings or to otherwise modify the physical form of the solid unit dosage form. For instance, tablets. pills, or capsules may be coated with gelatin, wax, shellac, or sugar and the like. A syrup or elixir may contain the active compound, sucrose or fructose as a sweetening agent, methyl and propylparabens as preservatives, a dye and flavoring such as cherry or orange flavor. Of course, any material used in preparing any unit dosage form should be pharmaceutically acceptable and substantially non-toxic in the amounts employed. In addition, the active compound may be incorporated into sustained-release preparations and devices.

Compounds and compositions of the invention, including pharmaceutically acceptable salts or analogs thereof can be administered intravenously, intramuscularly, or intraperitoneally by infusion or injection. Solutions of the active agent or its salts can be prepared in water, optionally mixed with a nontoxic surfactant. Dispersions can also be prepared in glycerol, liquid polyethylene glycols, triacetin, and mixtures thereof and in oils. Under ordinary conditions of storage and use, these preparations can contain a preservative to prevent the growth of microorganisms.

The pharmaceutical dosage forms suitable for injection or infusion can include sterile aqueous solutions or dispersions or sterile powders comprising the active ingredient which are adapted for the extemporaneous preparation of sterile injectable or infusible solutions or dispersions, optionally encapsulated in liposomes. The ultimate dosage form should be sterile, fluid and stable under the conditions of manufacture and storage. The liquid carrier or vehicle can be a solvent or liquid dispersion medium comprising, for example, water, ethanol, a polyol (for example, glycerol, propylene glycol, liquid polyethylene glycols, and the like), vegetable oils, nontoxic glyceryl esters, and suitable mixtures thereof. The proper fluidity can be maintained, for example, by the formation of liposomes, by the maintenance of the required particle size in the case of dispersions or by the use of surfactants. Optionally, the prevention of the action of microorganisms can be brought about by various other antibacterial and antifungal agents, for example, parabens, chlorobutanol, phenol, sorbic acid, thimerosal, and the like. In many cases, it will be preferable to include isotonic agents, for example, sugars, buffers or sodium chloride. Prolonged absorption of the injectable compositions can be brought about by the inclusion of agents that delay absorption, for example, aluminum monostearate and gelatin.

Sterile injectable solutions are prepared by incorporating a compound of the invention in the required amount in the appropriate solvent with various other ingredients enumerated above, as required, followed by filter sterilization. In the case of sterile powders for the preparation of sterile injectable solutions, the preferred methods of preparation are vacuum drying and the freeze drying techniques, which yield a powder of the active ingredient plus any additional desired ingredient present in the previously sterile-filtered solutions.

Useful dosages of the compounds and pharmaceutical compositions of the present invention can be determined by comparing their in vitro activity, and in vivo activity in animal models. Methods for the extrapolation of effective dosages in mice, and other animals, to humans are known to the art; for example, see U.S. Pat. No. 4,938,949.

The dose administered to a patient, particularly a human, in the context of the present invention should be sufficient to achieve a therapeutic response in the patient over a reasonable time frame, without lethal toxicity, and preferably causing no more than an acceptable level of side effects or morbidity. One skilled in the art will recognize that dosage will depend upon a variety of factors including the condition (health) of the subject, the body weight of the subject, kind of concurrent treatment, if any, frequency of treatment, therapeutic ratio, as well as the severity and stage of the pathological condition.

Mammalian species that benefit from the disclosed methods include, but are not limited to, primates, such as apes, chimpanzees, orangutans, humans, monkeys;

domesticated animals (e.g., pets) such as dogs, cats, guinea pigs, hamsters, Vietnamese pot-bellied pigs, rabbits, and ferrets; domesticated farm animals such as cows, buffalo bison, horses, donkey, swine, sheep, and goats; exotic animals typically found in zoos such as bear, lions, tigers, panthers, elephants, hippopotamus, rhinoceros, giraffes antelopes, sloth, gazelles, zebras, wildebeests, prairie dogs, koala bears, kangaroo opossums, raccoons, pandas, hyena, seals, sea lions, elephant seals, otters, porpoises dolphins, and whales. As used herein, the terms “subject” “host”, and “patient” are used interchangeably and intended to include such human and non-human mammalian species.

### MATERIALS AND METHODS FOR EXAMPLES 1-4

Animals. For this study, we have used single transgenic mice expressing human APP containing the Swedish mutation (K670N:M671L) (TgAPPswe) (4), and wild-type (WT) littermates. Mice were treated for two months before the behavioral studies. At weaning, the animals were genotyped from tail biopsies by means of an appropriate digest and polymerase chain reaction.

Western Blot. After two months of Sorafenib treatment (20 mg/kg/day), mice were sacrificed and cortex and hippocampus removed by dissection. Triton-soluble protein tissue extracts were separated by gradient SDS-PAGE 4-20%, transferred to nitrocellulose membranes, blocked with 5% skim milk in TBS-Tween 0.05%, and incubated with primary antibodies against pcRaf-1[Ser338], IκB-α, pNFκB[Ser276], APP (22C11), COX-2, and β-tubulin overnight. After washing, membranes were incubated with appropriate secondary antibodies for 1-2 hours. Immunoreactive bands were visualized using ECL.

Aβ toxicity assay. Primary rat cortical cells were incubated with 5 μM Aβ in the presence or absence of the cRaf-1 inhibitor, GW5074, for 48 hours. Total cell extracts were used for the analysis of Aβ-dependent NFκB phosphorylation by western blotting.

Cell viability. Cell viability was quantified by MTT and propidiurn iodide (PI) and calcein-AM staining assays. MTT assay measures the mitochondrial conversion of the tetrazolium salt MTT (3-(4,5-dimethylthiazol-2-yl)-2,5-diphenyl tetrazolium bromide) to formazan. To perform the MTT assay, the cell culture medium was replaced with medium containing MTT (0.5 mg/mL). Following 1-4 hours of incubation at 37° C., MTT formazan crystals were dissolved in DMSO and absorbance at 570 nm was measured. Absorbance values are a measure of cell viability. MIT results are expressed as percentage of vehicle treated controls. MIT assay was performed by measuring the levels of formazan after dissolving the precipitates in DMSO. The double calcein-AM and PI staining, involves staining with PI (a fluorescent nucleic acid dye used for the staining of dead cells) and calcein-AM a compound that inside of live brain cells, is converted to the green fluorescent compound calcein. After 30 minutes of incubation of the cells with these compounds, cells were washed and analyzed by fluorescence microscopy.

All patents, patent applications, provisional applications, and publications referred to or cited herein are incorporated by reference in their entirety, including all figures and tables, to the extent they are not inconsistent with the explicit teachings of this specification.

Following are examples that illustrate procedures for practicing the invention. These examples should not be construed as limiting. All percentages are by weight and all solvent mixture proportions are by volume unless otherwise noted.

### Example 1

### Raf-1 Inhibitor GW5074 Inhibited the Aβ-Dependent Increase in NFκB Phosphorylation

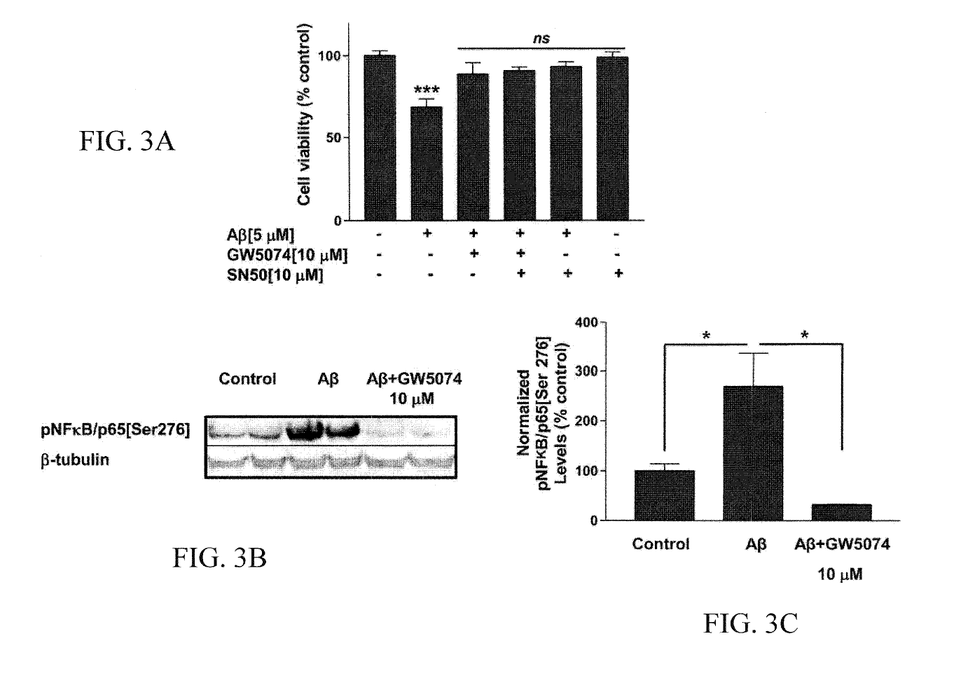

Cortical cells were cultured in Neurobasal/B27 media. After 7-10 days in vitro, cells were co-treated with 5 μM Aβ and the Raf inhibitor, GW5074. After 48 hours, cell viability was analyzed by western blot against pNFκB[Ser276] and β-tubulin (FIGS. 3B and 3C).

### Example 2

### Sorafenib Decreases the Levels of the Active Form of cRaf-1 in APPswe Brains

Post-nuclear fractions of cortex from wild type mice (n=5), APPswe (n=4), and Sorafenib treated APPswe n=5) mice were analyzed for cRaf-1 levels by western blot.

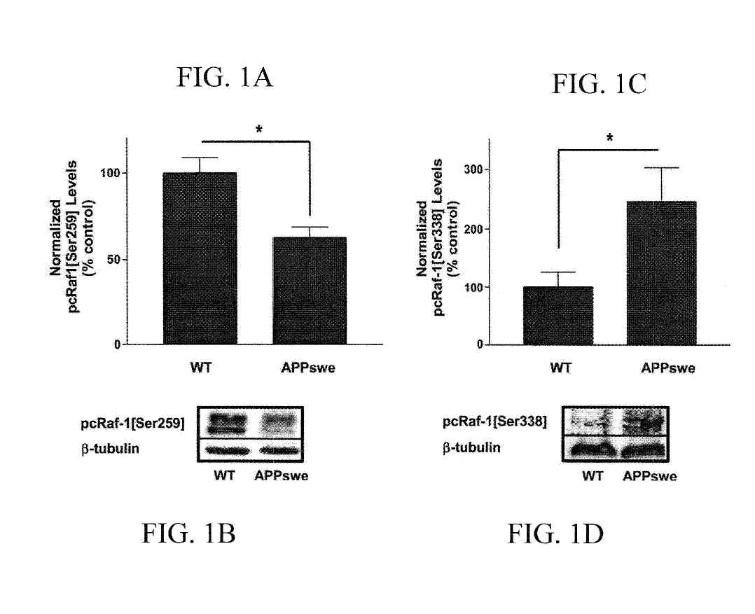

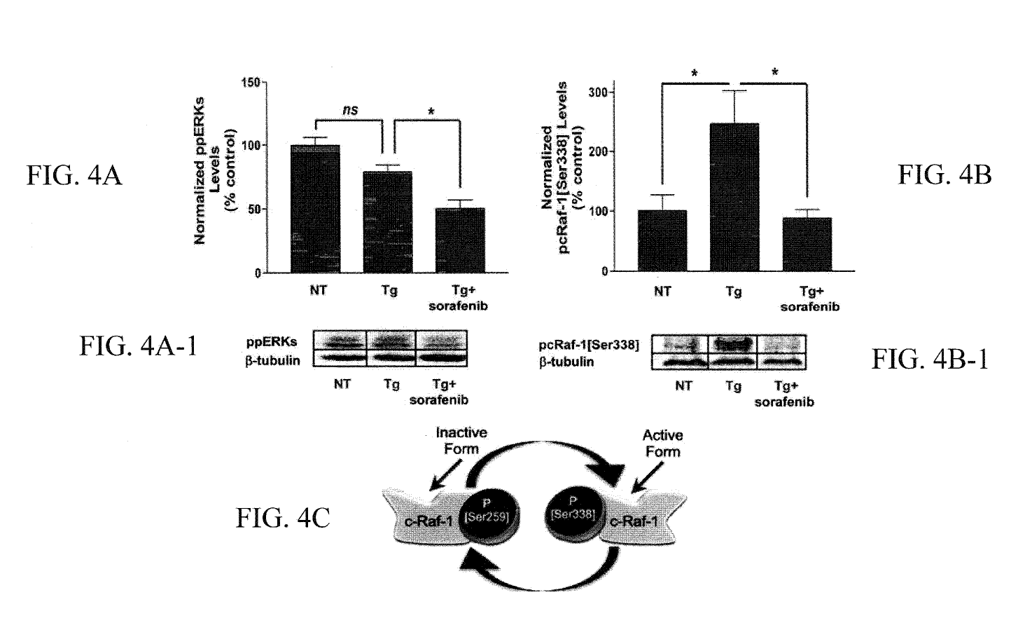

The histogram represents the levels of pcRaf-1[Ser338] normalized against β-tubulin. (**P<0.01). Western blot analysis of phospho-cRaf-1[Ser338] β-tubulin used as control (FIG. 4B-1).

### Example 3

### Sorafenib Inhibits the NFκB Signaling in the Cortex of Aged APPswe Mice

To study whether Sorafenib affected the NFκB pathway, 14-16 month-old APPswe mice and age-matched control littermates were treated for two months with Sorafenib (20 mg/kg/day) by gavage. After this time, mice were sacrificed and the levels of IκB-α (FIG. 3A), NFκB phosphorylated at serine 276 (FIG. 3B), Cox-2 (FIG. 3C), and APP (FIG. 3D) were analyzed by western blot. The histogram represents immunoreactivity values normalized against β-tubulin. *P<0.05, **P<0.01, ***P<0.001. Wt (n=5), APPswe (n=4), APPswe (n=5).

### Example 4

### Working Memory Task-Interference Test

Interference test details. Cognitive Interference Task. This task involves two radial arm water maze set-ups in two different rooms, and different sets of visual cues. The task requires animals to remember a set of visual cues, so that following interference with a different set of cues, the initial set of cues can be recalled to successfully solve the radial arm water maze task. A set of five behavioral measures were examined. Behavioral measures are: A1-A3 (Composite three-trial recall score from first 3 trials performed in RAWM “A”), “B” (proactive interference measure attained from a single trial in RAWM “B”), A4 (retroactive interference measure attained during a single trial in RAWM “A”), and “A5” (delayed-recall measure attained from a single trial in RAWM “A” following a 20 min delay between A4 and A5). As with the standard RAWM task, this interference task involves the platform location being changed daily to a different arm for both of the RAWM set-ups utilized, and different start arms for each day of testing for both RAWM set-ups. For A1 and B trials, the animal is initially allowed one minute to find the platform on their own before they are guided to the platform. Then the actual trial is performed in each case. As with the standard RAWM task, animals were given 60-s to find the escape platform, with the number of errors and escape latency recorded for each trial.

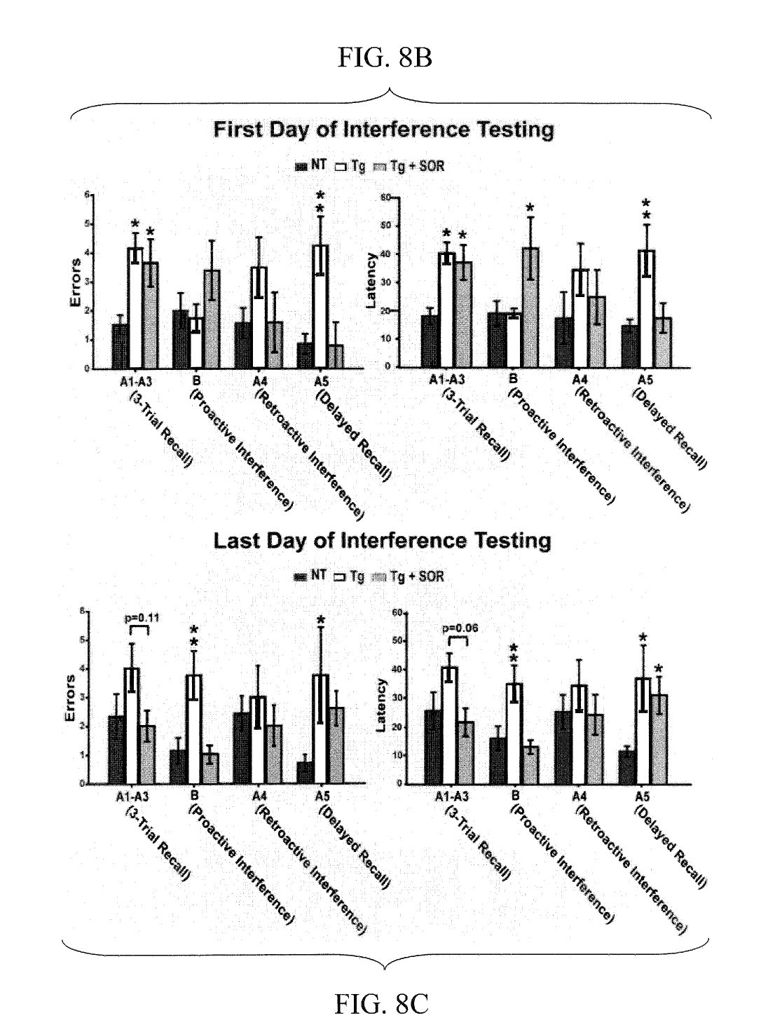

APPswe mice not treated with Sorafenib display a deficit in working memory expressed as a higher number of errors on this task (FIGS. 8B and 8C). Sorafenib produced a significant improvement in working memory in the APPswe mice compared with vehicle-treated APPswe mice (p<0.01). The transgenic mice did not present any motor or sensory differences with age-matched wild type mice.

The results obtained clearly show that APPswe mice presenting AD pathology and treated 2 months with sorafenib perform as well as wild type age-matched controls and significantly different from APPswe vehicle treated mice.

TABLE 1
Sorafenib activity on different protein kinases

| Kinase | IC₅₀ nm ± SD ( )* |
|---|---|
| cRaf-1 | 6 ± 37(7) |
| B-Raf | 22 ± 6(7) |
| VEGFR-2 | 38 ± 9(4) |
| Flt-3 | 58 ± 20(5) |
| c-Kit | 68 ± 21(3) |
| FGFR-1 | 580 ± 100(3) |
| ERK1, MEK1, EGFR, HER2, IGFR1, | >10,000 |
| cmet, PKB, PKA, cdk1, /cyclinB, PKCα, | |
| PKCγ, pim-1 | |

IC₅₀ mean ± standard deviation (SD) (n = number of trials) (Wilhelm et al., 2004)

### Example 5

A great deal of published evidence supports the concept that the accumulation of amyloid β-peptide (Aβ) in the brain induces synaptic dysfunction and neuronal cell death. The use of cultured neurons and transgenic AD animal models such as the APPswe mice has permitted the discovery of several molecular changes induced by Aβ toxicity and potential therapeutic targets against AD.

The neurochemical analyses of the brain of patients with AD showed that high levels of Aβ are accompanied by changes in several protein kinases involved in neuronal survival such as cAMP-dependent protein kinase A (PKA) (Liang et al. (2007)), extracellular regulated kinase (ERK) (Ferrer et al. (2001b)), and cRaf-1 (Mei et al. (2006)). cRaf-1 activity is controlled by phosphorylation at two sites; cRaf-1 is generally found to be inactive when phosphorylated at serine 259, and active when phosphorylated at serine 338 (Beeram et al. (2003); Kunnimalaiyaan and Chen (2006)). The dysregulation of cRaf-1 found in AD brains is coherent with a decrease in proteins that inhibit its activity, such as the Raf kinase inhibitor protein (RKIP) (George et al. (2006)) and PKA (Liang et al. (2008)), and an increase of proteins that stimulate PKA such as Ras (McShea et al. (1999)).

To investigate whether cRaf-1 dysregulation is a general mechanism of AD, we analyzed cRaf-1 expression and phosphorylation in the brains of aged APPswe mice. Since Raf overactivation is a molecular hallmark in brains exposed to high levels of Aβ, we investigated the effect of the Raf inhibitors GW5074 and ZM336372 on Aβ toxicity in cultured cortical cells. A previous study showed that these cRaf-1 inhibitors protected cerebellar neurons against other neurotoxins by a mechanism that required NFκB activity (Chin et al. (2004)). In the same report, acute treatment with GW5074 decreased neurodegeneration in a mouse model of Huntington's disease (Chin et al. (2004)). cRaf-1 stimulates NFκB activity by activating the regulator inhibitor κB (IKK) kinase (IKK) (Li and Sedivy (1993)). When activated, IKK induces the degradation of the inhibitor of NFκB, IκBα. Then, NFκB translocates into the nucleus where it stimulates the expression of amyloid β-precursor protein (APP) and β-secretase 1 (BACE1) (Bourne et al. (2007)). The Raf inhibitor GW5074 decreased the phosphorylation of NFκB at the activation site serine 276, suggesting that GW5074 is neuroprotective against Aβ toxicity by inhibiting NFκB.

Most chemicals including GW5074 (Chin et al. (2004)) and ZM336372 were purchased from Sigma Chemicals (St. Louis, Mo., USA) and all antibodies were purchased from Cell Signaling, Inc. (Beverly, Mass., USA) unless specified otherwise. SN50 was purchased from Calbiochem (San Diego, Calif., USA). Aβ₁₋₄₂ peptide was obtained from American Peptide (Sunnyvale, Calif., USA). Hoechst 33342, propidium iodide, dichlorodihydrofluorescein diacetate (CM-H₂DCFDA) and materials used for cell culture were obtained from Invitrogen (Carlsbad, Calif., USA).

The in vivo studies were performed using 16-18-month-old transgenic APPswe mice containing the Swedish mutation (K260N/M671L) and age-matched wild-type littermate mice. Mouse brains were removed after euthanasia by cervical dislocation, and the cortex dissected out and frozen at −80° C. until use. Mice were maintained on a 12 h dark and 12 h light cycle with ad libitum access to food and water. Animals were used in accordance with the National Institutes of Health guidelines for the use of experimental animals. Protocols were approved by the Institutional Animal Care and Use Committee of the University of South Florida, and Bay Pines and Tampa VA Healthcare Systems.

Embryonic rat cortical neurons were cultured following the protocol described previously (Brewer (1995)) with minor modifications. Briefly, cerebral cortices were obtained from BrainBits LLC (Springfield, Ill., USA). The brain tissues were dissociated by 0.05% (v/v) trypsin digestion and triturated. Cells (1250 cells/mm²) were plated in Neurobasal medium E, supplemented with 1 mM glutamax, and 2% B27 and plated onto 24-well plates coated with poly-D-lysine (0.1 mg/ml). The cell cultures were kept at 37° C. in a humidified incubator with 95% air/5% CO₂ until used.

After 7-10 days in vitro (DIV), the media was removed and the cortical cells were incubated with Neurobasal medium supplemented with B27 minus antioxidant (B27-AO; Invitrogen) for 2 h. Then cells were exposed to Aβ₁₋₄₂ oligomers in the presence or absence of GW5074 or ZM336372. Aβ₁₋₄₂ solution was prepared to obtain oligomers by dissolving 0.5 mg of Aβ₁₋₄₂ peptide in a solution of sodium hydroxide (1 mM) and an equal volume of phosphate-buffered saline (PBS), pH 7.4 (Invitrogen). The Aβ₁₋₄₂ solution was then diluted into the culture medium to reach a final concentration of 5 μM as previously described (Echeverria et al. (2005)). All assays were performed in triplicate and repeated at least three times.

Cell viability was quantified by the MTT assay that measures the mitochondrial conversion of the tetrazolium salt MTT (3-(4,5-dimethylthiazol-2-yl)-2,5-diphenyl tetrazolium bromide) to formazan as described (Brewer (1995)). Cells were incubated with MTT (0.5 mg/ml) for 1-3 h at 37° C.; MTT formazan crystals were dissolved in DMSO and absorbance at 570 nm was measured. Cell viability was also estimated with propidium iodide (PI) and calcein. 7 DIV cortical cells were incubated in 1 ml of media containing 1 μg/ml of PI and calcein-AM for 30 min at 37° C. After incubation, cells were washed with PBS and fluorescence was determined with a fluorescence microscope (Microscope LeicaDMI4000B, 20×, 40×). The changes in cell viability were determined by analyzing the number of calcein-AM (green, living) and PI (red nucleus, dead) cells in any experimental condition. At least 300 cells were analyzed by condition. Assays were measured in triplicate and repeated at least twice. Results are expressed as percentage of vehicle-treated controls.

Cortical cells (0.5×10⁶ cells/well) after 7 DIV and brain tissues from 16-18-month-old APPswe mice were disrupted by sonication in cold lysis buffer (Cell Signaling Technology, Beverly, Mass., USA) containing a complete protease inhibitor cocktail (Roche Molecular Biochemicals, Indianapolis, Ind., USA), 1 mM phenylmethyl sulphonyl fluoride (PMSF; Sigma, Saint Louis, Mich., USA), and phosphatase inhibitors (Sigma). After sonication, protein extracts were incubated on ice and centrifuged to 16,000×g for 30 min at 4° C. and supernatants were used for Western blot analysis.

Equal amounts of protein were separated by sodium dodecyl sulphate-polyacrylamide gel electrophoresis (SDS-PAGE) and transferred to nitrocellulose membranes (BA83 0.2 μm; Bio-Rad). The membranes were blocked in phosphate buffered saline with 0.05% Tween-20 (PBST) containing 10% skim milk. Membranes were incubated with primary antibodies in PBST with 3% dry milk overnight at 4° C. and with secondary antibodies for 2 h. Rabbit polyclonal antibodies were directed against pcRaf-1 (Ser259) (1:500) and pcRaf-1 (Ser338) (1:500) (Cell Signaling, Inc., CA, USA), and pNFκB/p65 (Ser276) (1:500) (GenScript Corporation, NJ, USA). A monoclonal mouse antibody, anti β-tubulin (Promega, Wis., USA), was used as a control for protein loading. The bands were detected using ECL detection kit (ECL, Pharmacia Biotech, Piscataway, N.J., USA), visualized using the KODAK Image Station 440CF and analyzed using the NIH Image) software. All data was normalized against tubulin immunoreactivity and expressed as percentage of control values.

We and others have found that the active form of cRaf-1 is upregulated in the cortex of human AD brains (Mei et al. (2006)). To investigate whether cRaf-1 dysregulation was a common characteristic of AD brains, we analyzed by Western blot the levels of the inactive form phosphorylated at serine 259 and the active form phosphorylated at serine 338 of cRaf-1 in the cortex of APPswe mice. We found a significant decrease in the inactive form of cRaf-1 (mean±SEM: 63±6%; P=0.02, n=3) (37% reduction) (FIG. 1A) and a significant increase in the levels of the active form (mean±SEM: 247±56%; P=0.038, n=4) (147% increase) in the cortex of 16-18-month-old APPswe mice compared to wild-type control littennates (mean±SEM: 100±26%; n=5) (FIG. 1B). The characteristic double bands migrated with a relative molecular weight of 75-100 kDa.

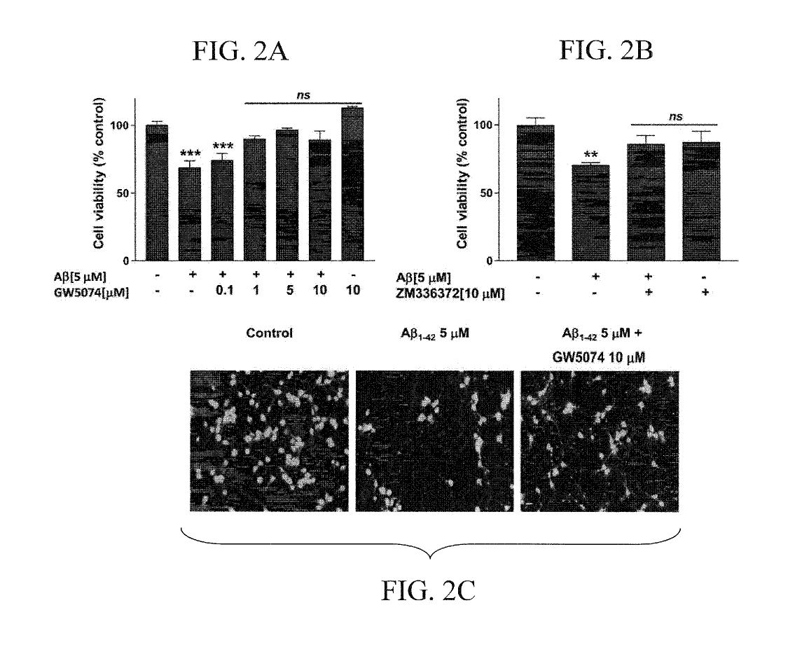

To investigate the effect of cRaf-1 inhibitors on neuronal survival after Aβ₁₋₄₂ toxicity, cortical cells were co-treated with Aβ peptide 5 μM and different concentrations of either GW5074 or ZM336372, and after 48 h cell viability was analyzed. The co-treatment of neurons with both Raf inhibitors was protective against AI3₁₋₄₂ toxicity. The co-treatment of cells with the Raf inhibitors increased cell survival from 73% (5 μM Aβ) to 93% (5 μM Aβ+GW5074, 10 μM) and from 70% (5 μM Aβ) to 86% (5 μM Aβ+ZM336372 10 μM) of control levels as estimated by MTT assay (FIGS. 2A and 2B). A similar pattern of neuroprotection was observed using calcein-AM (live cells) and PI (dying cells) staining. The number of PI stained cells after Aβ exposure decreased dramatically when cells were co-treated with 10 μM GW5074 (FIG. 2C).

It has been found that NFκB/p65 expression is elevated in brain cells surrounding senile plaques in the brains accumulating Aβ (Kaltschmidt et al. (1999)). Moreover, the inhibition of NFκB is protective against Aβ neurotoxicity (Chen et al. (2005)). To investigate whether NFκB activity was involved in the beneficial actions of GW5074, we studied the effect of SN50 on GW5074 activity. We found that the treatment of neurons with the NFκB inhibitor SN50, alone or in the presence of 10 μM GW5074, induced similar levels of neuroprotection against Aβ toxicity as GW5074 (FIG. 3A).

To determine the effect of GW5074 on the Aβ-induced activation of NFκB, we investigated NFκB/p65 phosphorylation at serine 276 in cortical cells treated with: (a) PBS; (b) 5 μM Aβ; and (c) 5 μM Aβ plus 10 μM GW5074. We found that cells treated with 5 μM Aβ showed an almost three-fold increase in NFκB/p65 phosphorylation at serine 276 (mean±SEM: 270±65%) compared to vehicle-treated control cells (mean±SEM: 100±14%). Cells co-treated with 10 μM GW5074 and 5 μM Aβ presented a nine-fold decrease in NFκB/p65 phosphorylation in comparison to Aβ treated cells (FIG. 3C).

We investigated cRaf-1 expression and activity in the cortex of APPswe mice and the effect of the cRaf-1 inhibitors GW5074 and ZM336372 on Aβ-dependent activation of NFκB and neurotoxicity. The cRaf-1 inhibitors provided neuroprotection against Aβ toxicity through a mechanism that was mimicked by the NFκB inhibitor SN50. Supporting the idea that Raf inhibitors are neuroprotective by inhibiting NFκB, we found that GW5074 inhibited the Aβ-dependent activation of NFκB. Since we obtained neuroprotection using two different cRaf-1 inhibitors, we postulate that Raf inhibition underlies its beneficial actions. Coherent with the inhibition of cRaf-1 by GW5074,we observed a decrease in the activation by phosphorylation of NFκB at serine 276 in cultured cortical neurons. Our results suggest that cRaf-1 hyperactivity is a molecular hallmark of AD and a key factor inducing Aβ synthesis. One of the obvious explanations of the abnormal activation of cRaf-1 in the brain of AD patients is the increased activity of the upstream activator of cRaf-1, Ras (Ferrer et al. (2001a)). It has been hypothesized that Ras can promote apoptosis in the brain by triggering the re-entrance of neurons into the cell cycle (Ferrer et al. (2001b)).

Moreover, abnormal cRaf-1 activation can be due to the decrease of endogenous inhibitors such as PKA (Kim et al. (2001)) and the Raf kinase inhibitor protein (RKIP) (Keller et al. (2004)). Since PKA activity is downregulated in AD brains, this may be another factor causing cRaf-1 dysregulation. In relation to the mechanism underlying the beneficial effects of Raf inhibitors, we found that the protection conferred by GW5074 against Aβ paralleled a decrease of NFκB/p65 activation. It is well known that after its activation by phosphorylation at serine 338 (Dhillon et al. (2007)), cRaf-1 stimulates NFκB activity (Liu et al. (2001)). NFκB/p65, one of the more abundant NFκB subunits in the central nervous system, is unregulated in the brains of patients suffering from several neurodegenerative conditions including Parkinson's disease, Amyotrophic lateral sclerosis (Barger et al. (2005); Ghosh et al. (2007)) and AD (Kaltschmidt et al. (1997)). It is feasible that the inhibition of NFκB activation by Raf inhibitors can be also beneficial in these conditions. Such a mechanism would be in line with numerous reports showing that inhibition of NFκB reduces Aβ toxicity (Valerio et al. (2006)) and its production (Paris et al. (2007)).

NFκB is inhibited by many neuroprotective compounds against AD (Nam (2006)) such as resveratrol, quercetin, Ginkgo Biloba and curcumin. We propose that a similar mechanism is underlying the observed neuroprotective activity of Raf inhibitors against Aβ toxicity. For example, the actions of quercetin against cancer are attributed to the inhibition of Raf and the resultant decrease in NFκB activity (Lee et al. (2008)). The expected outcome of NFκB inhibition includes a decrease in the expression of the Aβ synthesizing enzymes such as BACE1 (Bourne et al. (2007)). Several Raf inhibitors have been developed for the treatment of cancer. One of these inhibitors, sorafenib, which is already approved by the Food and Drug Administration as a treatment for kidney (Hutson (2007)) and liver cancers (Lang (2008)), is particularly interesting because of its minimal side effects in humans (Takimoto and Awada (2008)) and its ability to be administered orally to the patients.

### Example 6

Alzheimer's disease (AD) is a neurodegenerative condition characterized by a progressive loss of memory. These pathological characteristics are accompanied by signs of neuroinflammation, such as gliosis and an increase in brain levels of amyloid β peptide (Aβ), pro-inflammatory factors such as the cytokines, vascular endothelial growth factor (VEGF) (Tarkowski et al. (2002); Lopez-Lopez et al. (2007)), tumor necrosis factor α (TNF α) (Tarkowski et al. (2002)), cRaf-1, nuclear factor kappa B (NF-κB), cyclooxygenase-2 (Cox-2) (Kaltschmidt et al. (1997); Mattson and Camandola (2001); Echeverria et al. (2008)), and the inducible nitric oxide synthase (iNOS). Here, we investigated the effect of chronic treatment of APPswe mice (transgenic [Tg]) with the multi-kinase inhibitor sorafenib (NEXAVAR, Onyx-Bayer) on the expression of these pro-inflammatory factors and on working memory abilities.

Cox-2 is a rate limiting enzyme in prostanoid synthesis that is synaptically induced and expressed by excitatory neurons at postsynaptic sites in the cortex of rats (Kaufmann et al. (1996)) and has been found to be unregulated in AD brains (Yagami (2006)). Cox-2 upregulation correlates with a faster decline of cognitive abilities in AD patients (Melnikova et al. (2006); Yagami (2006)). More importantly, Cox-2 inhibition prevented working memory deficits in Tg mice (Cakala et al. (2007)) and ameliorated the Aβ-induced inhibition of hippocampal long-term potentiation, a cellular model of memory (Kotilinek et al. (2008)).

Here, we investigated the effect of cRaf-1 inhibition on AD pathology using NEXAVAR, the tosylate salt of sorafenib. NEXAVAR is a drug that is already approved by the Food and Drug Administration (FDA) to treat advanced clear-cell renal cell carcinoma patients (Bellmunt et al. (2007)). NEXAVAR is orally available and presents minor side effects in humans (Takimoto and Awada (2008)). Sorafenib, the main component of NEXAVAR, is an orally active multi-kinase inhibitor that crosses the blood-brain barrier (Kane et al. (2006)) and selectively targets Raf, vascular endothelial growth factor receptor 2/3 (VEGFR), platelet-derived growth factor receptor (PDGFR)-β, FLT-3, and c-Kit.

Although sorafenib is a multi-kinase inhibitor, it is mainly a highly effective inhibitor of cRaf-1 (IC₅₀=6 nM). We have previously reported that the highly specific cRaf-1 inhibitor, GW5074, protected primary neurons against Aβ toxicity and inhibited NF-κB activation (Echeverria et al. (2008)). NF-κB is a transcription factor broadly expressed in the nervous system, including neurons as well as glia, and a downstream target of cRaf-1, which mediates inflammatory responses (Mattson and Camandola (2001)). NF-κB consists of several subunits that mainly include p50 and p65 in the brain (Chen et al. (2005); Collister and Albensi (2005); Townsend and Pratico (2005); Kim et al. (2006); Liu et al. (2007); Paris et al. (2007)). Supporting the idea that NF-κB inhibition is one of the mechanisms underlying the neuroprotection conferred by cRaf-1 inhibition, we found that the NF-κB inhibitor, SN50, mimicked the neuroprotective effect of GW5074 against Aβ toxicity (Echeverria et al. (2008)). Our results are in agreement with previous evidence showing that the inhibition of NF-κB is neuroprotective against Aβ toxicity in vitro (Paris et al. (2007)).

In resting conditions, NF-κB is sequestered in the cytoplasm by binding to its inhibitors, IκBs (Mattson and Camandola (2001)). One of the main mechanisms of NF-κB activation in neurons is the degradation of one of those inhibitors, IκBα (Tergaonkar et al. (2003)). IκBα is degraded after phosphorylation by the IκBα kinase complex, which is activated by cRaf-1. Freed NF-κB translocates into the nucleus, where it activates the expression of genes containing the κB sites. Since cRaf-1 controls the degradation of IκB-α, we investigated whether the chronic treatment with the c-Raf-1 inhibitor, sorafenib, inhibited the degradation of IκBα in the cortex of aged APPswe mice. We found that chronic treatment with sorafenib increased the levels of IκBα and inhibited the activation of NF-κB in the brain of these AD mice. The stimulation of NF-κB activity by cRaf-1 controls the expression of several factors relevant to AD, including iNOS and Cox-2. Consistent with a role of the cRaf-1/NF-κB pathway in controlling the expression of these factors in AD brains, we observed a pronounced decrease in the expression of iNOS and Cox-2 in the brains of the sorafenib-treated APPswe mice. These beneficial signal transduction effects were associated with restoration of cognitive abilities in the sorafenib-treated AD mice.

### Experimental Procedures

### Reagents

Most chemicals were purchased from Sigma Chemicals (St. Louis, Mo., USA) and all antibodies used in this study were purchased from Cell Signaling, Inc. (Beverly, Mass., USA) unless specified otherwise. Sorafenib (4-(4-phenoxy)-N2-methylpyridine-2 carboxamide 4-methylbenzenesulfonate, commercial name NEXAVAR) was obtained from Bay Pines VA Healthcare System Research Pharmacy.

### Animals

All mice were derived from a cross between heterozygous mice carrying the mutant APPK670N, M671L gene (APPsw) with heterozygous PS1 (Tg line 6.2) mice to obtain mice consisting of amyloid beta peptide precursor protein (APP)/PS1, APPsw, PS1, and non-tg (NT) genotypes. Each mouse had a mixed background of 56.25% C57, 12.5% B6, 18.75% SJL, and 12.5% Swiss-Webster. This Tg AD model was originally developed by insertion of the human APP695 construct with the “Swedish” double mutation and hamster prion protein cosmid vector into the host (Hsiao et al. (1996)). After weaning and genotyping, only male APPswe and NT mice were selected for these studies. All mice were maintained on a 12-h light/dark cycle with ad libitum access to rodent chow and water. Animals were used in accordance with the National Institutes of

Health guidelines for the use of experimental animals. Protocols were approved by the Institutional Animal Care and Use Committee of the University of South Florida, Bay Pines and Tampa VA Healthcare Systems. We used the minimal number of animals possible in order to obtain results that allow a reliable statistical analysis of the data.

### General Protocol

Two separate studies were done to investigate the effects of sorafenib on APPswe and NT mice. In Study I, APPswe and NT mice were evaluated in the radial arm water maze (RAWM) task of working memory for 8 days immediately prior to initiation of treatment. Then, beginning at 15-16 months of age, half of the animals in each genotype were started on daily sorafenib treatment via gavage (20 mg/kg/day) and the other half given daily PBS vehicle treatment. Each of the four genotypic/treatment groups consisted of four to seven mice. At 6 weeks into treatment, mice were re-evaluated in the RAWM task for 9 days, followed several days later by 6 days of testing in a novel cognitive interference task (with continuing treatment). At 17-18 months of age, mice were euthanized and brain tissues microdissected out into cortex and hippocampus; tissue samples were frozen immediately on dry ice and stored at −80° C. until use.

In Study II, APPswe and NT mice at 13-15 months of age were started on either daily sorafenib or vehicle treatment, with four to five mice in each of the four genotypic/treatment groups. At 4 months into treatment (e.g., at 17-19 months of age), all mice were euthanized and their brains processed as in Study I.

### Behavioral Tasks

RAWM. For the RAWM task of spatial working memory (Arendash et al. (2001b); Arendash et al. (2007); Ethell et al. (2006)), an aluminum insert was placed into a 100 cm circular pool to create six radially distributed swim arms emanating from a central circular swim area (Arendash et al. (2001a); Ethell et al. (2006)). An assortment of 2-D and 3-D visual cues surrounded the pool. The number of errors prior to locating which one of the six swim arms contained a submerged escape platform (9 cm diameter) was determined in each of five trials every day. There was a 20-min time delay between the 4th trial (T4; final acquisition trial) and 5th trial (T5; memory retention trial). The platform location was changed daily to a different arm, with different start arms for each of the five trials semi-randomly selected from the remaining five swim amis. During each trial (60 s maximum), the mouse was returned to that trial's start arm upon swimming into an incorrect arm and the number of seconds required to locate the submerged platform was recorded. If the mouse did not find the platform within a 60-s trial, it was guided to the platform for the 30-s stay. The number of errors and escape latency during trials 4 and 5 are both considered indexes of working memory and are temporally similar to the standard registration/recall testing of specific items used clinically in evaluating AD patients.

Cognitive interference task. This task involves two RAWM setups in two different rooms, and three different sets of visual cues. The task requires animals to remember a set of visual cues, so that following interference with a different set of cues, the initial set of cues can be recalled to successfully solve the RAWM task. A set of four behavioral measures was examined through six successive daily trials. Behavioral measures were A1-A3 (Composite three-trial recall score from first three trials performed in RAWM “A”), “B” (proactive interference measure attained from a single trial in RAWM “B”), A4 (retroactive interference measure attained during a single trial in RAWM “A”), and “A5” (delayed-recall measure attained from a single trial in RAWM “A” following a 20 min delay between A4 and A5). As with the standard RAWM task, this interference task involves the platform location being changed daily to a different arm for both of the RAWM setups utilized, and different start arms for each day of testing for both RAWM setups. For A1 and B trials, the animal is initially allowed 1 min to find the platform on its own before being guided to the platform. Then the actual trial is performed in each case. Also, between each trial, animals were placed into a Y-maze apparatus for 30 seconds as an inter-trial interfering experience. As with the standard RAWM task, animals were given 60 s to find the escape platform for each trial, with the number of errors and escape latency recorded for each trial.

### Neurochemical Analysis

Sample preparation. Mouse brains were quickly removed after euthanasia by cervical dislocation, dissected, frozen and maintained at −80° C. until use. Brain tissues were collected, powdered in liquid nitrogen, and disrupted by sonication in cold lysis buffer, containing 20 mM Tris-HCl (pH 7.5), 150 mM NaCl, 1 mM Na2EDTA, 1 mM EGTA, 1% Triton, 2.5 mM sodium pyrophosphate, 1 mM beta-glycerophosphate, 1 mM Na3VO4 and 1 μg/ml leupeptin (Cell Signaling Technology) containing complete protease inhibitor cocktail (Roche Molecular Biochemicals, Indianapolis, Ind., USA), 1 mM phenylmethanesulphonyl fluoride (Sigma, St. Louis, Mo., USA) and phosphatase inhibitors. After sonication, cell extracts were incubated on ice for 30 mM and centrifuged to 16,000×g for 30 min at 4° C., and supernatant was isolated and used for Western blotting analysis.

Western blotting. Protein concentration of supernatants was measured by Bio-Rad protein assay (Bio-Rad, Hercules, Calif., USA), and equal amounts of protein (40-80 μg) were separated by sodium dodecyl sulphate-polyacrylamide gel electrophoresis (SDS-PAGE) and transferred to nitrocellulose membranes (BA83 0.2 μm; Bio-Rad). The membranes were blocked in Tris-buffered saline with 0.05% Tween 20 (TTBS) containing 10% dry milk for 45 min. After blocking, membranes were incubated with primary antibodies in TTBS with 3% dry milk overnight at 4° C. and with secondary antibodies for 1 h. Rabbit polyclonal antibodies were directed against pcRaf-1[Ser338] (1:500), phospho-pErk1/2 kinases (Thr202/tyr204) (ppErk1/2) (Cell Signaling Technology, Inc., CA, USA) (1:1000), IκB-α (1:100) (C21, Santa Cruz Biotechnology, Santa Cruz, Calif., USA) and pNFκB/p65[Ser276] (1:100) (GenScript Corporation, NJ, USA). Monoclonal mouse antibody directed against Cox-2 (1:1000) (Cayman, Mich., USA) and iNOS (1:250) (Cell Signaling) were used. In addition, a mAb against β-tubulin (Promega, Wis., USA) was used to control protein sample loading and transfer efficiency. The immunoreactive bands were visualized using ECL kit (ECL, Pharmacia Biotech, Piscataway, N.J., USA), the KODAK Image Station 440CF and analyzed using the NIH ImageJ software. The immunoreactivity values were normalized to the N-terminal segment of β-tubulin, then converted and expressed as a percent of control values.

Determination of Aβ levels. The levels of Aβ₁₋₄₀ and Aβ₁₋₄₂ were quantified in the brain tissues by ELISA.

Soluble Aβ levels. Brain tissues were homogenized according to the protocol described by Schmidt et al. (2005a; 2005b). Homogenates were extracted with diethylamine (DEA) and supernatants obtained were stored at −80° C. and assayed to determine soluble Aβ levels by using an ELISA kit obtained from Signet Laboratories and according to the manufacturer's recommendations.

Total Aβ levels. Brain tissues were prepared by sonication of the samples in 5 M guanidine HCl, and 50 mM Tris-HCl. pH 8.0. After sonication, samples were incubated for 3 h at room temperature and centrifuged at 16,000×g for 20 min at 4° C. The supernatants were stored at −80° C. For analysis, the supernatants were diluted using PBS with 5% BSA and 0.03% Tween-20 supplemented with 1× protease inhibitor cocktail (Roche, USA) and further used to determine total Aβ levels using a commercial ELISA kit (Invitrogen, Carlsbad, Calif., USA), according to the manufacturer's recommendations.

### Protein Kinase A (PKA) Assay

The activity of PKA in the brain tissues of mice was measured in total brain extracts using a non-radioactive PKA kinase activity ELISA assay kit (Assay Designs, Ann Arbor, Mich., USA) according to the manufacturer's instructions. This ELISA measures the phosphorylation of a synthetic peptide substrate by PKA in the brain sample. The values were expressed as a percentage of wild type control values considered as 100% enzymatic activity. All determinations were performed at least in quadruplicate and repeated twice.

### Statistical Analysis

For statistical analysis of RAWM data, the 8 days of pre-treatment testing (four 2-day blocks) or the 9 days of during-treatment testing (three 3-day blocks) were evaluated for individual blocks, as well as over all blocks, using one-way ANOVAs. Thereafter, post hoc pair-by-pair differences between groups were resolved with the Fisher LSD (least significant difference) test. For statistical analysis of cognitive interference, data from first and last days of testing were utilized since we have shown these days to be particularly insightful of performance in this task. One-way ANOVAs were employed for each of the four behavioral measures analyzed, followed by post hoc Fisher's LSD test to determine group differences. All group comparisons involving neurochemical measures involved one-way ANOVAs with post hoc Fisher's LSD test. To compare the mean differences between two groups Student's t-test was also used.

### Results

Sorafenib is a multikinase inhibitor that targets the protein kinase cRaf-1, which has been found to be overactivated in the brain of AD patients (Mei et al. (2006)). To investigate the relevance of the hyperactivation of cRaf-1 and its impact on memory loss, we treated aged APPswe mice (Tg) presenting cognitive impairment with sorafenib and studied their working memory abilities and cerebral levels of proinflammatory factors, such as NF-κB. Consistent with the idea that sorafenib crosses the blood-brain barrier, we found changes in the levels of the protein factors, such as cRaf-1, and several NF-κB-inducible pro-inflammatory signaling proteins in the brain of sorafenib-treated Tg mice compared to age-matched wild type non-transgenic (NT) littermates as detailed below.

Upregulation of the Active Form of cRaf-1 is Suppressed by Two Months of Treatment with Sorafenib in the Brain of APPswe Mice

No changes in cRaf-1 or ERK phosphorylation were observed in wild type NT mice treated with sorafenib when compared to untreated NT mice. Also, there were no differences in cortical levels of the active faun of ERKs in Tg control mice compared to NT group's, which was considered as 100% level of immunoreactivity (FIG. 4A). However, levels of cRaf-1 phosphorylated at Ser 338 (cRaf-1 Ser338]) revealed a statistically significant upregulation of the active form of pcRaf-1[Ser338] (FIG. 4C) in the cortex of Tg control mice (FIG. 4B). In sharp contrast, Tg mice treated with sorafenib (20 mg/kg) for two months presented a significant reduction (29% decrease) in the levels of ppERK compared to control Tg mice (FIG. 4A). Likewise, a highly significant 158% decrease in pcRaf-1[Ser 338] was present in Tg mice treated with sorafenib when compared with control Tg mice.

### Sorafenib Treatment Decreases NF-κB Signaling in the Cortex of APPswe Mice

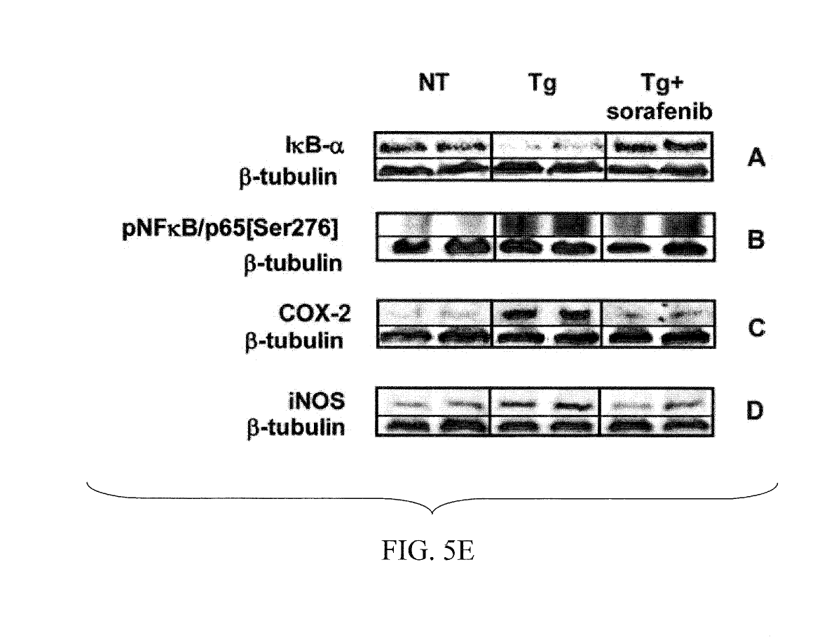

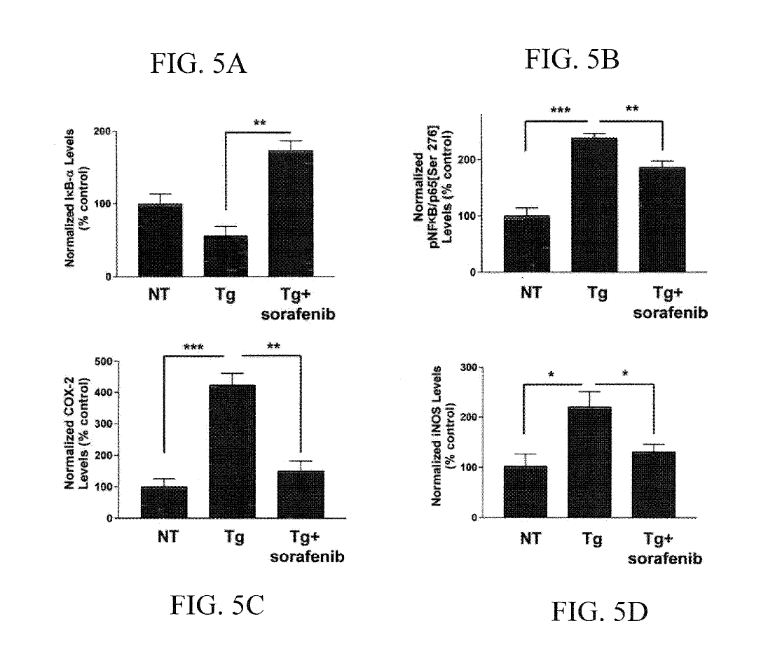

cRaf-1 signaling modulates the activation of NE-κB by stimulating the degradation of the NF-κB inhibitor IκBα (von Bulow et al. (2007)). Previously, we reported that the cRaf-1 inhibitor, GW5074, inhibited NF-κB signaling in cortical neurons subjected to Aβ toxicity (Echeverria et al. (2008)). Here, we show that two months sorafenib-treatment increased IκBα and, consistent with this increase in the NF-κB inhibitor, a longer four month treatment decreased the activation of NF-κB, expressed as a reduction of NF-κB phosphorylated at serine 276 (Ser 276) in the brains of Tg mice. In these studies, 13-15-month-old Tg and NT mice were treated continuously with sorafenib or vehicle, after which mice were euthanized at 15-17 (Study I) or 17-19 (Study II) months of age, and analyzed for molecular changes in cell signaling factors and Aβ levels in the brain. Specifically, we analyzed the expression of the cell signaling factors IκBα. iNOS, Cox-2, and pNF-κB[Ser276] in the detergent-soluble fractions of the cortex of the mice by Western blot. We found that Tg mice at 15-17 months of age showed an impressive increase in the levels of several markers of neuroinflammation, such as Cox-2, iNOS, and the active form of NF-κB (FIGS. 5A-5E). The chronic treatment of the mice with sorafenib corrected or lessened these protein abnormalities as Tg mice treated with sorafenib for four months showed significantly lower levels of Cox-2, iNOS, and pNF-κB[Ser276].

When Tg mice were treated for two months, higher levels of IκBα compared to control Tg mice were found in the cortex (FIG. 5A). More specifically, we found that Tg mice showed a 44% decrease in IκBα levels in the cortex (FIG. 5A) compared to NT mice. However, Tg mice treated with sorafenib showed 117% higher levels of IκBα protein expression compared to control Tg mice. Indeed, Tg mice treated with sorafenib exhibited expression levels of IκBα that were higher than those present in NT mice.

As previously described by other reports (Collister and Albensi (2005)), AD pathology was accompanied by an activation of NF-κB as detected by analyzing NF-κB phosphorylation at the activation site, Ser 276. We observed a significant increase in the levels of pNF-κB[Ser276] in the cortex of Tg mice compared to NT mice—an increase that was significantly reduced by sorafenib treatment (FIG. 5B). No differences in pNF-κB[Ser276] levels were observed between untreated wild type mice and wild type mice treated with sorafenib (data not shown).

In line with an upregulation of NF-κB activity and the expression of genes regulated by this neuroinflammatory factor, such as Cox-2, we found that Tg mice presented higher levels of this proinflammatory enzyme in the cortex compared to NT mice (FIG. 5C). Tg mice showed a significant threefold increase in Cox-2 in the cortex compared to NT mice. By sharp contrast, sorafenib-treated Tg mice presented substantially lower levels of Cox-2 in the cortex compared to control Tg mice. Finally, sorafenib-treated Tg mice presented an 81% lower expression of iNOS in the cortex than control Tg mice (FIG. 5D), a reduced expression that essentially normalized iNOS levels in Tg mice to iNOS levels in NT mice. No differences in iNOS and Cox-2 levels were observed between untreated wild type mice and wild type mice treated with sorafenib (data not shown).

### Sorafenib Treatment did not Reduce the Levels of AP in the Brain of APPswe Mice

To determine the effect of sorafenib treatment on brain levels of insoluble Aβ in the aged Tg mice, we analyzed total Aβ levels in the cortex of Tg mice treated with sorafenib or vehicle for 2 months and 4 months. We found that the sorafenib-treated Tg mice did not show a decrease in the levels of total Aβ₄₀ and Aβ₄₂ compared to control Tg mice, although a trend for reduced total Aβ levels was evident for Tg mice treated with sorafenib (Table 2). Since no significant changes in total Aβ (even after 4 months of treatment) were observed, we analyzed whether sorafenib decreased the levels of soluble Aβ in the hippocampus of the Tg mice. We found that Tg mice treated with sorafenib for four months had similar levels of soluble Aβ₄₀ and Aβ₄₂ as Tg controls in the hippocampus (Table 2).

TABLE 2
Sorafenib treatment did not reduce the levels of Aβ in the brain of APPswe mice

| | Tg | | Tg + Sorafenib | |
|---|---|---|---|---|
| | Study 1 | Study 2 | Study 1 (2 mo. treatment) | Study 2 (4 mo. treatment) |
| Aβ₄₀ [pg/mg] | | | | |
| ^{A}Total levels | 14,840 ± 1,072 | 25,000 ± 14,470 | 8,835 ± 2,517 ns, P = 0.1322 | 36,140 ± 9.281 ns, P = 0.5193 |
| ^{B}Soluble levels | N/A | 361.0 ± 90.10 | N/A | 267.0 ± 104.0 ns, P = 0.5293 |
| Aβ₄₂ [pg/mg] | | | | |
| ^{C}Total levels | 29,300 ± 6,230 | 71,570 ± 17,090 | 22,570 ± 5,946 ns, P = 0.4568 | 71,980 ± 26,210 ns, P = 0.9914 |
| ^{D}Soluble levels | N/A | 119.1 ± 49.51 | N/A | 82.80 ± 28.54 ns, P = 0.5242 |

Aβ levels represent the concentration of amyloid peptides in the cortex (A, C) and hippocampus (B, D) of the mice expressed as picogram by milligram (pg/mg) of brain tissue. Total peptide levels correspond to the levels found in the supernatant of cortical tissues homogenized in 5 M guanidinium chloride. Soluble Aβ levels correspond to the levels found in the supernatant of hippocampal tissues of the mice homogenized in the Triton-soluble fractions. ns, Not significant (P>0.05).

### Sorafenib Increased PKA Activity and CREB Phosphorylation in Aged APPswe Mice

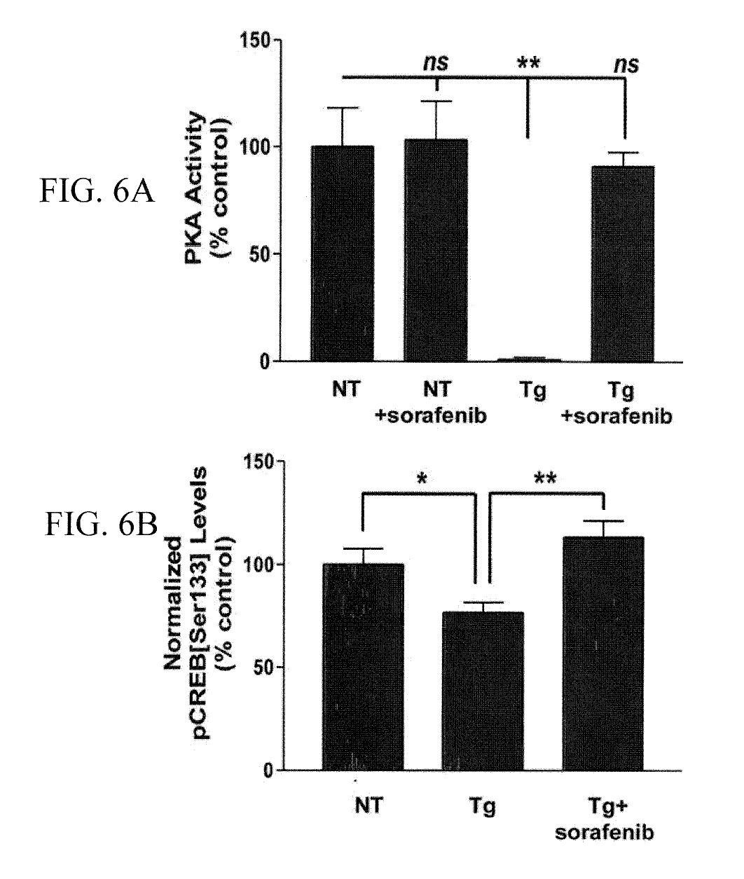

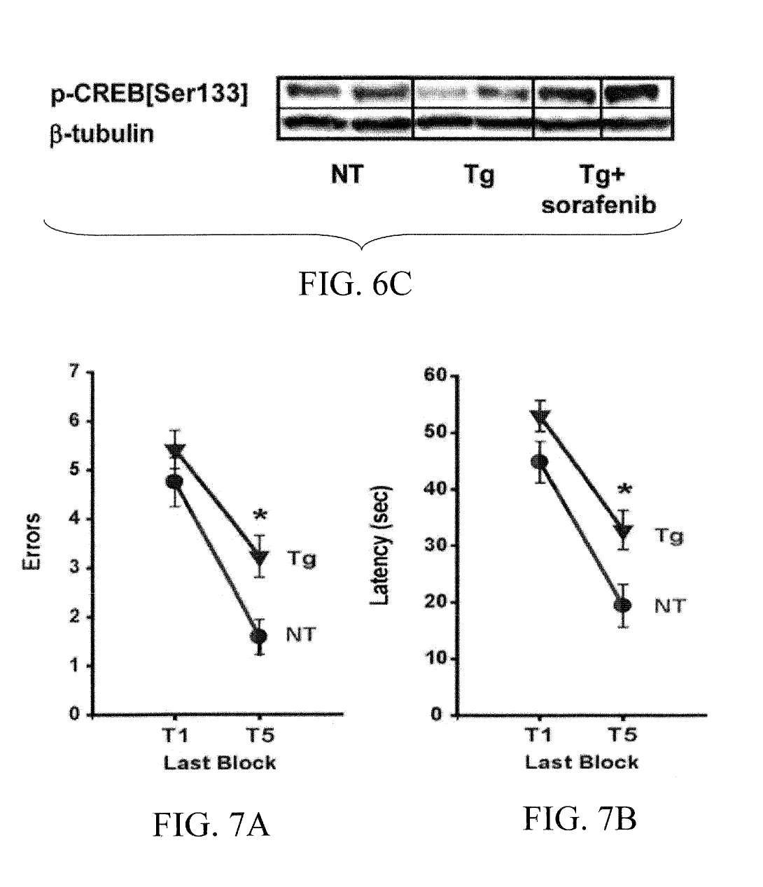

The analysis of PKA activity in brain extracts from mice treated with sorafenib for four months indicated a pronounced reduction in its activity in the cortex of Tg control mice compared to age-matched wild-type littermate mice (FIG. 6A). Sorafenib treatment for four months restored PKA activity in Tg mice to values similar to those observed in NT mice. Since PKA stimulates CREB phosphorylation at serine 133 (Ser 133), we also investigated CREB phosphorylation in the cortex of sorafenib-treated Tg, vehicle-treated Tg and NT mice. The results obtained showed that Tg mice presented a significant decrease in CREB phosphorylation at Ser 133 in comparison with age-matched wild type littermate mice, considered 100% (FIG. 6B). In sharp contrast, treatment of Tg mice with sorafenib significantly increased CREB phosphorylation in the cortex of Tg mice to levels present in NT mice.

Two Months of Treatment with Sorafenib Restored Working Memory in Aged APPswe Mice

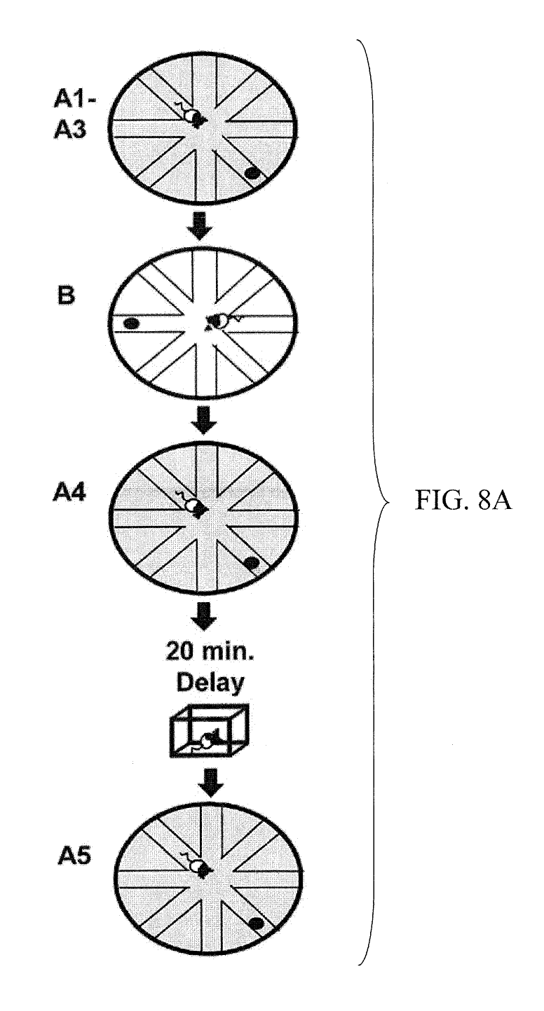

To test the hypothesis that sorafenib can be beneficial in vivo against AD pathology, we tested whether the chronic treatment with the cRaf-1 inhibitor improved the cognitive abilities of APPswe mice. First, to confirm that Tg mice were cognitively impaired prior to sorafenib treatment, animals were evaluated in the RAWM task of working memory. During the last block of this pre-treatment testing (FIGS. 7A and 7B), Tg mice performed similar to NT controls during the naive trial 1 (T1) of testing, wherein they discover that day's platform location for the first time. By contrast, Tg mice made significantly more errors (FIG. 7A) and had significantly higher escape latencies (FIG. 7B) during working memory trial 5 (T5). Nonetheless, because both NT and Tg groups improved their performance across trials (T1 vs. T5), the working memory impairment of Tg mice was only modest in extent.

RAWM testing during treatment revealed that both NT and NT+sorafenib groups performed exceptionally well and, although both Tg and Tg+sorafenib groups were not quite as good as NT groups, neither Tg group exhibited the robust impairment typical of Tg mice of this age and genotype. For example, T5 performance across all blocks for Tg mice should be between three and four errors. However, T5 errors over all blocks of RAWM testing were NT 0.9±0.4; NT-Fsorafenib 1.1±0.2; Tg 1.9±0.3; and Tg+sorafenib 1.9±0.2. Given this good performance of Tg mice in our standard RAWM task, follow-up testing involved our much more demanding cognitive interference task.

There were no differences in performance between NT and NT+sorafenib groups for any measure of cognitive interference testing, so their near-identical data were combined into a single NT group for comparison with Tg and Tg+sorafenib groups. During the first day of interference testing (FIG. 8B), both Tg and Tg+sorafenib mice were impaired for the three-trial recall measure (A1-A3) compared to NT mice. By the final day of testing (FIG. 8C), sorafenib-treated Tg mice performed identically to NT mice on this three-trial recall measure, both in reference to errors and escape latency. For the important proactive interference measure (B), Tg+sorafenib mice actually showed strong perseveration of RAWM A's platform location during the first day of testing by showing a relatively high escape latency to locate RAWM B's location (FIG. 8B). However, by the last day of testing (FIG. 8C), Tg controls exhibited a robust impairment in proactive interference that was reversed by sorafenib treatment. Tg+sorafenib mice performed significantly better than Tg controls and identically to NT mice. For the retroactive interference trial (A4), NT and Tg groups performed similarly for both the first and last day of interference testing, indicating no effect of genotype or sorafenib treatment on this measure (FIGS. 8B and 8C). By contrast, the 20-min delayed recall trial (A5) revealed a substantial impairment of Tg controls during the first day of testing (FIG. 8B), while sorafenib-treated Tg mice were significantly better and no different from NT mice. By the final day of testing, however, the beneficial effects of sorafenib on A5 were not evident (FIG. 8C).

To summarize the behavioral testing results, sorafenib did not improve upon the good performance of both NT and Tg mice in standard RAWM testing. However, when challenged by the more difficult cognitive interference task, impairments in delayed recall (early testing) and proactive interference (late testing) became manifest in Tg mice. Both of these cognitive impairments in Tg mice were eliminated by sorafenib treatment.

It should be understood that the examples and embodiments described herein are for illustrative purposes only and that various modifications or changes in light thereof will be suggested to persons skilled in the art and are to be included within the spirit and purview of this application and the scope of the appended claims. In addition, any elements or limitations of any invention or embodiment thereof disclosed herein can be combined with any and/or all other elements or limitations (individually or in any combination) or any other invention or embodiment thereof disclosed herein, and all such combinations are contemplated with the scope of the invention without limitation thereto.

### REFERENCES

U.S. Pat. No. 4,816.567
U.S. Pat. No. 4,938,949
U.S. Pat. No. 5,225,539
U.S. Pat. No. 5,270,163
U.S. Pat. No. 5,411,749
U.S. Pat. No. 5,475,096
U.S. Pat. No. 5,567,588
U.S. Pat. No. 5,580,737
U.S. Pat. No. 5,707,796
U.S. Pat. No. 5,763,177
U.S. Pat. No. 5,840,867
U.S. Pat. No. 6,011,577
U.S. Pat. No. 6,506,559
U.S. Pat. No. 7,056,704
U.S. Pat. No. 7,078,196
U.S. Pat. No. 7,232,086
U.S. Pat. No. 7,282,564
U.S. Pat. No. 7,307,071
U.S. Pat. No. 7,365,058
U.S. Pat. No. 7,491,829
U.S. Pat. No. 7,538,095
U.S. Pat. No. 7,566,716
U.S. Published Application No. 2009/0215835
International Published Application No. WO 00/042012
International Published Application No. WO 00/41698
International Published Application No. WO 06/034796
International Published Application No. WO 06/034797
International Published Application No. WO 2004/064733
International Published Application No. WO 91/00906
International Published Application No. WO 91/10741
International Published Application No. WO 92/01047
International Published Application No. WO 92/03917
International Published Application No. WO 92/03918
International Published Application No. WO 93/05796
International Published Application No. WO 93/12227
International Published Application No. WO 94/04667
International Published Application No. WO 94/25585
International Published Application No. WO 95/17085
International Published Application No. WO 96/07754

Arendash G W, Garcia M E, Costa D A, Cracchiolo J R, Wefes I M, Potter H (2004) Environmental enrichment improves cognition in aged Alzheimer's transgenic mice despite stable beta-amyloid deposition. Neuroreport 15:1751-1754.
Arendash G W, Gordon M N, Diamond D M, Austin L A, Hatcher J M, Jantzen P et al. (2001a) Behavioral assessment of Alzheimer's transgenic mice following long-term Abeta vaccination: task specificity and correlations between Abeta deposition and spatial memory. DNA Cell Biol 20:737-744.
Arendash G W, Jensen M T, Salem N Jr, Hussein N, Cracchiolo J, Dickson A et al. (2007) A diet high in omega-3 fatty acids does not improve or protect cognitive performance in Alzheimer's transgenic mice. Neuroscience 149:286-302.
Arendash G W, King D L, Gordon M N, Morgan D, Hatcher J M, Hope C E, Diamond D M (2001 b) Progressive, age-related behavioral impairments in transgenic mice carrying both mutant amyloid precursor protein and presenilin-1 transgenes. Brain Res 891:42-53.
Arendash G W, Mori T, Cao C. Mamcarz M, Runfeldt M, Dickson A, Rezai-Zadeh K, Tan J, Citron B A, Lin X, Echeverria V, Potter H. (2009) Caffeine reverses cognitive impairment and decreases brain Aβ levels in aged Alzheimer's mice. J Alzheimers Dis, 17(3):661-80.
Autier J, Escudier B, Wechsler J, Spatz A, Robert C (2008) Prospective study of the cutaneous adverse effects of sorafenib, a novel multikinase inhibitor. Arch Dermatol 144:886-892.
Barbas et al. Proc. Nat'l Acad. Sci. USA 89:4457-4461, 1992
Barger, S. W., A. M. Moerman, X. Mao (2005) Molecular mechanisms of cytokineinduced neuroprotection: NFkappaB and neuroplasticity, Curr. Pharm. Des. 11:985-998.
Beeram, M., A. Patnaik, E. K. Rowinsky (2003) Regulation of c-Raf-1: therapeutic implications, Clin. Adv. Hematol. Oncol. 1:476-481.
Beidler et al., J. Immunol. 141:4053-4060, 1988
Bellmunt J, Montagut C, Albiol S, Caries J, Maroto P, Orsola A (2007) Present strategies in the treatment of metastatic renal cell carcinoma: an update on molecular targeting agents. BJU Int 99:274-280.
Better et al., Science 240:1041-1043, 1988
Bird et al., Science 242:423-426, 1988
Bourne, K. Z., D. C. Ferrari, C. Lange-Dohna, S. Rossner, T. G. Wood, J. R. Perez-Polo (2007) Differential regulation of BACE1 promoter activity by nuclear factor-kappaB in neurons and glia upon exposure to beta-amyloid peptides, J. Neurosci. Res. 85:1194-1204.
Brewer, G. J. (1995) Serum-free B27/neurobasal medium supports differentiated growth of neurons fromthe striatum, substantia nigra, septum, cerebral cortex, cerebellum, and dentate gyrus, J. Neurosci. Res. 42:674-683.
Brosnan C F, Battistini L, Raine C S, Dickson D W, Casadevall A, Lee S C (1994) Reactive nitrogen intermediates in human neuropathology: an overview. Dev Neurosci 16:152-161.
Bruggeman et al. (1991) Eur. J. Immunol. 21:1323-1326
Bruggeman et al. Year Immunol. 7:33-40, 1993
Cakala M, Malik A R, Strosznajder J B (2007) Inhibitor of cyclooxygenase-2 protects against amyloid beta peptide-evoked memory impailnlent in mice. Pharmacol Rep 59:164-172.
Chen, J., Y. Zhou, S. Mueller-Steiner, L. F. Chen, H. Kwon, S. Yi, L. Mucke, L. Gan (2005) SIRT1 protects against microglia-dependent amyloid-beta toxicity through inhibiting NF-kappaB signaling, J. Biol. Chem. 280:40364-40374.
Cheng A L, Kang Y K, Chen Z, Tsao C J, Qin S, Kim J S, Luo R, Feng J, Ye S et al. (2009) Efficacy and safety of sorafenib in patients in the Asia-Pacific region with advanced hepatocellular carcinoma: a phase III randomised, double-blind, placebo-controlled trial. Lancet Oncol, 10(1):25-34.
Chin, P. C., L. Liu, B. E. Morrison, A. Siddiq, R. R. Ratan, T. Bottiglieri, S. R. D'Mello (2004) The c-Raf inhibitor GW5074 provides neuroprotection in vitro and in an animal model of neurodegeneration through a MEK-ERK and Akt-independent mechanism, J. Neurochem. 90:595-608.
Choi et al. Nature Genet. 4:117-123, 1993
Collister K A, Albensi B C (2005) Potential therapeutic targets in the NF-kappaB pathway for Alzheimer's disease. Drug News Perspect 18:623-629.
Costa D A, Cracchiolo J R, Bachstetter A D, Hughes T F, Bales K R, Paul S M, Mervis R F, Arendash G W, Potter H (2007) Enrichment improves cognition in AD mice by amyloid-related and unrelated mechanisms. Neurobiol Aging 28:831-844.
Dhillon A S, Pollock C, Steen H, Shaw P E, Mischak H, Kolch W (2002) Cyclic AMP-dependent kinase regulates Raf-1 kinase mainly by phosphorylation of serine 259. Mol Cell Biol 22:3237-3246.
Dhillon, A. S., A. von Kriegsheim, J. Grindlay, W. Kolch (2007) Phosphatase and feedback regulation of Raf-1 signaling, Cell Cycle 6:3-7.
Dooley A, Gao B, Bradley N, Abraham D J, Black C M, Jacobs M, Bruckdorfer K R (2006) Abnormal nitric oxide metabolism in systemic sclerosis: increased levels of nitrated proteins and asymmetric dimethylarginine. Rheumatology (Oxford) 45:676-684.
Echeverria V, Burgess S, Gamble-George J, Arendash G W, Citron B A (2008) Raf inhibition protects cortical cells against beta-amyloid toxicity. Neurosci Lett, 444(1):92-6.
Echeverria V, Cuello A C (2002) Intracellular A-beta amyloid: a sign for worse things to come? Mol Neurobiol 26:299-316.
Echeverria, V., A. Clerman, S. Dore (2005) Stimulation of PGE receptors EP2 and EP4 protects cultured neurons against oxidative stress and cell death following beta-amyloid exposure, Eur. J. Neurosci. 22:2199-2206.
Eisen T, Oudard S, Szczylik C, Gravis G, Heinzer H, Middleton R, Cihon F et al. (2008) Sorafenib for older patients with renal cell carcinoma: subset analysis from a randomized trial. J Natl Cancer Inst 100:1454-1463.
Elser C, Siu L L, Winquist E, Agulnik M, Pond G R, Chin S F, Francis P, Cheiken R et al. (2007) Phase II trial of sorafenib in patients with recurrent or metastatic squamous cell carcinoma of the head and neck or nasopharyngeal carcinoma. J Clin Oncol 25:3766-3773.
Escudier B (2007) Sorafenib [corrected] in kidney cancer. Ann Oncol 18 (Suppl 9):ix90-ix93
Estevez A G, Spear N, Manuel S M, Barbeito L, Radi R, Beckman J S (1998) Role of endogenous nitric oxide and peroxynitrite formation in the survival and death of motor neurons in culture. Prog Brain Res 118:269-280.
Ethell D W, Shippy D, Cao C, Cracchiolo J R, Runfeldt M, Blake B, Arendash G W (2006) Abeta-specific T-cells reverse cognitive decline and synaptic loss in Alzheimer's mice. Neurobiol Dis 23:351-361.
Ferrer, I., R. Blanco, M. Carmona, B. Puig (2001a) Phosphorylated c-MYC expression in

Alzheimer disease, Pick's disease, progressive supranuclear palsy and corticobasal degeneration, Neuropathol. Appl. Neurobiol. 27:343-351.
Ferrer, I., R. Blanco, M. Carmona, R. Ribera, E. Goutan, B. Puig, M. J. Rey et al. (2001b) Phosphorylated map kinase (ERK1, ERK2) expression is associated with early tau deposition in neurones and glial cells, but not with increased nuclear DNA vulnerability and cell death, in Alzheimer disease, Pick's disease, progressive supranuclear palsy and corticobasal degeneration, Brain Pathol. (Zurich, Switzerland) 11:144-158.
George, A. J., R. M. Holsinger, C. A. McLean, S. S. Tan, H. S. Scott, et al. (2006) Decreased phosphatidylethanolamine binding protein expression correlates with Abeta accumulation in the Tg2576 mouse model of Alzheimer's disease, Neurobiol. Aging 27:614-623.
Ghosh, A., A. Roy, X. Liu, J. H. Kordower, E. J. Mufson, D. M. Hartley, et al. (2007) Selective inhibition of NF-kappaB activation prevents dopaminergic neuronal loss in a mouse model of Parkinson's disease, Proc. Natl. Acad. Sci. U.S.A. 104:18754-18759.
Green, L. L. et al. Nature Genet. 7:13-21, 1994
Griffin R J, Moloney A, Kelliher M, Johnston J A, Ravid R, Dockery P, O'Connor R et al. (2005) Activation of Akt/PKB, increased phosphorylation of Akt substrates and loss and altered distribution of Akt and PTEN are features of Alzheimer's disease pathology. J Neurochem 93:105-117.
Griffiths et al. EMBO J 12:725-734, 1993
Gupta-Abramson V, Troxel A B, Nellore A, Puttaswamy K, Redlinger M et al. (2008) Phase II trial of sorafenib in advanced thyroid cancer. J Clin Oncol 26:4714-4719.
Harlow and Lane, (1988) Antibodies: A Laboratory Manual ed., Cold Spring Harbor Press
Heneka M T, Wiesinger H, Dumitrescu-Ozimek L, Riederer P. Feinstein. D L et al. (2001) Neuronal and glial coexpression of argininosuccinate synthetase and inducible nitric oxide synthase in Alzheimer disease. J Neuropathol Exp Neurol 60:906-916.
Hsiao K, Chapman P, Nilsen S, Eckman C, Harigaya Y, Younkin S, Yang F, Cole G (1996) Correlative memory deficits. Abeta elevation, and amyloid plaques in transgenic mice. Science 274:99-102.
Huang Y, Liu F, Grundke-Iqbal I, Iqbal K, Gong C X (2005) NF-kappaB precursor, p105, and NF-kappaB inhibitor, IkappaBgamma, are both elevated in Alzheimer disease brain. Neurosci Lett 373:115-118.
Hull M, Lieb K, Fiebich B L (2000) Anti-inflammatory drugs: a hope for Alzheimer's disease? Expert Opin Invest Drugs 9:671-683.
Huston et al., Proc. Natl. Acad. Sci. USA 85:5879-5883, 1988
Hutson, T. E. (2007) Safety and tolerability of sorafenib in clear-cell renal cell carcinoma: a Phase III overview, Exp. Rev. Anticancer Therapy 7:1193-1202.
Jones et al., Nature 321:552-525, 1986
Kaltschmidt, B., M. Uherek, B. Volk, P. A. Baeuerle, C. Kaltschmidt (1997) Transcription factor NF-kappaB is activated in primary neurons by amyloid beta peptides and in neurons surrounding early plaques from patients with Alzheimer disease, Proc. Natl. Acad. Sci. U.S.A. 94:2642-2647.
Kaltschmidt, B., M. Uherek, H. Wellmarm, B. Volk, C. (1999) Kaltschmidt, Inhibition of NF-kappaB potentiates amyloid beta-mediated neuronal apoptosis, Proc. Natl. Acad. Sci. U.S.A. 96:9409-9414.
Kamel-Reid et al. Science 242:1706, 1988
Kane R C, Farrell A T, Saber H, Tang S, Williams G, Jee J M, Liang C, Booth B et al. (2006) Sorafenib for the treatment of advanced renal cell carcinoma. Clin Cancer Res 12:7271-7278.
Kaufmann W E, Worley P F, Pegg J, Bremer M, Isakson P (1996) COX-2, a synaptically induced enzyme, is expressed by excitatory neurons at postsynaptic sites in rat cerebral cortex. Proc Natl Acad Sci USA 93:2317-2321.
Keller, E. T., Z. Fu, M. Brennan (2004) The role of Raf kinase inhibitor protein (RKIP) in health and disease, Biochem. Pharmacol. 68:1049-1053.
Khazak, V., Astsaturov, I., Serebriiskii, I., Golemis, E. (2007) Selective Raf inhibition in cancer therapy. Expert Opin Ther Targets 11(12):1587-1609.
Kim Y A, Lim S Y, Rhee S H, Park K Y, Kim C H, Choi B T, Lee S J, Park Y M, Choi Y U (2006) Resveratrol inhibits inducible nitric oxide synthase and cyclooxygenase-2 expression in beta-amyloidtreated C6 glioma cells. Int J Mol Med 17:1069-1075.
Kim, S. H., A. C. Nairn, N. Cairns, G. Lubec (2001) Decreased levels of ARPP-19 and PKA in brains of Down syndrome and Alzheimer's disease, J. Neural Transm. Suppl. 263-272.
King A J, Wireman R S, Hamilton M, Marshall M S (2001) Phosphorylation site specificity of the Pak-mediated regulation of Raf-1 and cooperativity with Src. FEBS Lett 497:6-14.
Kong L N, Zuo P P, Mu L, Liu Y Y, Yang N (2005) Gene expression profile of amyloid beta protein-injected mouse model for Alzheimer disease. Acta Pharmacol Sin 26:666-672.
Kotilinek L A, Westerman M A, Wang Q, Panizzon K, Lim G P, Simonyi A, Lesne S et (2008) Cyclooxygenase-2 inhibition improves amyloid-betamediated suppression of memory and synaptic plasticity. Brain 131:651-664.
Kunnimalaiyaan, M. and H. Chen (2006) The Raf-1 pathway: a molecular target for treatment of select neuroendocrine tumors? Anticancer Drugs 17:139-142.
Lang, L. (2008) FDA approves sorafenib for patients with inoperable liver cancer, Gastroenterology 134:379.
Lee, K. W., N. J. Kang, Y. S. Heo, E. A. Rogozin, A. Pugliese, M. K. Hwang et al. (2008) Raf and MEK protein kinases are direct molecular targets for the chemopreventive effect of quercetin, a major flavonol in red wine, Cancer Res. 68:946-955.
Li Q, Verma I M (2002) NF-kappaB regulation in the immune system. Nat Rev Immunol 2:725-734.
Li, S J. M. Sedivy (1993) Raf-1 protein kinase activates the NF-kappa B transcription factor by dissociating the cytoplasmic NF-kappa B-I kappa B complex, Proc. Natl. Acad. Sci. U.S.A. 90:9247-9251.
Liang, Y. Q., X. T. Huang, X. C. Tang (2008) Huperzine A reverses cholinergic and monoaminergic dysfunction induced by bilateral nucleus basalis magnocellularis injection of beta-amyloid peptide (1-40) in rats, Cell. Mol. Neurobiol, 28(1):87-101.
Liang, Z., F. Liu, I. Grundke-Iqbal, K. Iqbal, C. X. Gong (2007) Down-regulation of cAMP-dependent protein kinase by over-activated calpain in Alzheimer disease brain, J. Neurochem. 103 :2462-2470.
Liu et al., (1987a) J. Immunol. 139:3521-3526
Liu et al., (1987b) PNAS 84:3439-3443
Liu Q, Zhang J, Zhu H, Qin C, Chen Q, Zhao B (2007) Dissecting the signaling pathway of nicotine-mediated neuroprotection in a mouse Alzheimer disease model. FASEB J 21:61-73.
Liu, Q., J. Fan, M. McMahon, A. M. Prince, P. Zhang (2001) Role of the oncogenic Raf-1 in orchestration of discrete nuclear factor-kappaB-activating pathways, Mol. Cell. Biol. Res. Commun. 4:381-389.
Llovet J M, Ricci S, Mazzaferro V, Hilgard P, Gane E, Blanc J F, de Oliveira A C et al. (2008) Sorafenib in advanced hepatocellular carcinoma. N Engl J Med 359:378-390.
Lonberg, N. et al. Nature 368:856-859, 1994
Lopez-Lopez C, Dietrich M O, Metzger F, Loetscher H, Torres-Aleman I (2007) Disturbed cross talk between insulin-like growth factor I and AMP-activated protein kinase as a possible cause of vascular dysfunction in the amyloid precursor protein/presenilin 2 mouse model of Alzheimer's disease. J Neurosci 27:824-831.
Ma Q L, Yang F, Caton F, Ubcda O J, Hansen J E, Weisbart R H, Beech W et al. (2008) p21-activated kinase-aberrant activation and translocation in Alzheimer disease pathogenesis. J Biol Chem 283:14132-14143.
Marks et al J. Mol. Biol. 222:581-597, 1991
Matenia D, Griesshaber B, Li X Y, Thiessen A, Johne C. Jiao J, Mandelkow E et al. (2005) PAK5 kinase is an inhibitor of MARK/Par-1, which leads to stable microtubules and dynamic actin. Mol Biol Cell 16:4410-4422.
Mattson M P, Camandola S (2001) NF-kappaB in neuronal plasticity and neurodegenerative disorders. J Clin Invest 107:247-254.
McCune et al. Science 241:1632-1639, 1988
McGeer P L (2000) Cyclooxygenase-2 inhibitors: rationale and therapeutic potential for Alzheimer's disease. Drugs Aging 17:1-11.
McKee A C, Carreras I, Hossain L, Ryu H, Klein W L, Oddo S, La Ferla F M et al. (2008) Ibuprofen reduces Abeta, hyperphosphorylated tau and memory deficits in Alzheimer mice. Brain Res 1207:225-236.
McPhie D L, Coopersmith R, Hines-Peralta A, Chen Y, Ivins K J, Manly S P et al. (2003) DNA synthesis and neuronal apoptosis caused by familial Alzheimer disease mutants of the amyloid precursor protein are mediated by the p21 activated kinase PAK3. J Neurosci 23:6914-6927.
McShea, A., D. A. Zelasko, J. L. Gerst, M. A. Smith (1999) Signal transduction abnormalities in Alzheimer's disease: evidence of a pathogenic stimuli, Brain Res. 815:237-242.
Mei, M., B. Su, K. Harrison, M. Chao, S. L. Siedlak, L. A. Previll, L. Jackson et al. (2006) Distribution, levels and phosphorylation of Raf-1 in Alzheimer's disease, J. Neurochem. 99:1377-1388.
Melnikova T, Savonenko A, Wang Q, Liang X, Hand T, Wu L, Kaufmann W E et al. (2006) Cycloxygenase-2 activity promotes cognitive deficits but not increased amyloid burden in a model of Alzheimer's disease in a sex-dimorphic pattern. Neuroscience 141:1149-1162.
Minami H, Kawada K, Ebi H, Kitagawa K, Kim Y I, Araki K, Mukai H, Tahara M et al. (2008) Phase I and pharmacokinetic study of sorafenib, an oral multikinase inhibitor, in Japanese patients with advanced refractory solid tumors. Cancer Sci 99:1492-1498.
Morrison, S. L. et al. Proc. Natl. Acad. Sci. USA 81:6851-6855, 1994
Morrison, S. L., Science 229:1202-1207, 1985
Nam, N. H. (2006) Naturally occurring NF-kappaB inhibitors, Mini Rev. Med. Chem. 6:945-951.
Nathan C, Calingasan N, Nezezon J, Ding A, Lucia M S, La Perle K, Fuortes M et al. (2005) Protection from Alzheimer's-like disease in the mouse by genetic ablation of inducible nitric oxide synthase. J Exp Med 202:1163-1169.
Nguyen T V, Galvan V, Huang W, Banwait S, Tang H, Zhang J, Bredesen D E (2008) Signal transduction in Alzheimer disease: p21-activated kinase signaling requires C-terminal cleavage of APP at Asp664. J Neurochem 104:1065-1080.
Nishimura et al., Canc. Res. 47:999-1005, 1987
Oddo S, Caccamo A, Kitazawa M, Tseng B P, La Ferla F M (2003) Amyloid deposition precedes tangle formation in a triple transgenic model of Alzheimer's disease. Neurobiol Aging 24:1063-1070.
Oi et al., BioTechniques 4:214, 1986
Ophir G, Amariglio N. Jacob-Hirsch J, Elkon R, Rechavi G, Michaelson D M (2005) Apolipoprotein E4 enhances brain inflammation by modulation of the NF-kappaB signaling cascade. Neurobiol Dis 20:709-718.
Paris, D., N. Patel, A. Quadros, M. Linan, P. Bakshi, G. Ait-Ghezala, M. Mullan (2007) Inhibition of Abeta production by NF-kappaB inhibitors, Neurosci. Lett. 415:11-16.
Richly H, Henning B F, Kupsch P, Passarge K, Grubert M, Hilger R A et al. (2006) Results of a phase I trial of sorafenib (BAY 43-9006) in combination with doxorubicin in patients with refractory solid tumors. Ann Oncol 17:866-873.
Safarinejad M R (2008) Safety and efficacy of sorafenib in patients with castrate resistant prostate cancer: a phase II study. Urol Oncol, in press, corrected proof, available online 12 Sep. 2008.
Salminen A, Suuronen T, Kaarniranta K (2008) ROCK, PAK, and Toll of synapses in Alzheimer's disease. Biochem Biophys Res Commun 371:587-590.
Schmidt S D, Jiang Y, Nixon R A, Mathews P M (2005a) Tissue processing prior to protein analysis and amyloid-beta quantitation. Methods Mol Biol 299:267-278.
Schmidt S D, Nixon R A, Mathews P M (2005b) ELISA method for measurement of amyloid-beta levels. Methods Mal Biol 299:279-297.
Shaw et al., J. Natl. Cancer Inst. 80:1553-1559, 1988
Shinkai et al. Cell 68:855-868, 1992
Spanopoulou. Genes & Development 8:1030-1042, 1994
Strunaberg D, Awada A, Hirte H, Clark J W, Seeber S, Piccart P, Hofstra E et al. (2006) Pooled safety analysis of BAY 43-9006 (sorafenib) monotherapy in patients with advanced solid tumours: is rash associated with treatment outcome? Eur J Cancer 42:548-556.
Strumberg D, Clark J W, Awada A, Moore M J, Richly H, Hendlisz A, Hirte H W el al. (2007) Safety, pharmacokinetics, and preliminary antitumor activity of sorafenib: a review of four phase I trials in patients with advanced refractory solid tumors. Oncologist 12:426-437.
Sun et al. PNAS 84:214-218, 1987
Takimoto, C. H. and A. Awada (2008) Safety and anti-tumor activity of sorafenib (Nexavar) in combination with other anti-cancer agents: a reviewof clinical trials, Cancer Chemother. Pharmacal. 61:535-548.
Tanaka S, Takehashi M, Matoh N, Iida S, Suzuki I, Futaki S, Hamada H, Masliah E et al. (2002) Generation of reactive oxygen species and activation of NF-kappaB by non-Abeta component of Alzheimer's disease amyloid. J Neurochem 82:305-315.
Tarkowski E, Issa R, Sjogren M, Wallin A, Blennow K, Tarkowski A, Kumar P (2002) Increased intrathecal levels of the angiogenic factors VEGF and TGF-beta in Alzheimer's disease and vascular dementia. Neurobiol Aging 23:237-243.
Tergaonkar V, Bottero V, Ikawa M, Li Q, Verma I M (2003) IkappaB kinase-independent IkappaBalpha degradation pathway: functional NF-kappaB activity and implications for cancer therapy. Mol Cell Biol 23:8070-8083.
Townsend K P, Pratico D (2005) Novel therapeutic opportunities for Alzheimer's disease: focus on nonsteroidal anti-inflammatory drugs. FASEB J 19:1592-1601.
Tuaillon et al. PNAS 90:3720-3724,1993
Unger C, Hedberg M M, Mustafiz T, Svedberg M M, Nordberg A (2005) Early changes in Abeta levels in the brain of APPswe transgenic mice—implication on synaptic density, alpha7 neuronal nicotinic acetylcholine- and N-methyl-D-aspartate receptor levels. Mol Cell Neurosci 30:218-227.
Valerio, A., F. Boroni, M. Benarese, Sarnico, V. Ghisi, L. G. Bresciani et al. (2006) NF-kappaB pathway: a target for preventing beta-amyloid (Abeta)-induced neuronal damage and Abeta42 production, Eur. J. Neurosci. 23:1711-1720.
Verhoeyan et al., Science 239:1534,1988
Verma I M, Stevenson J K, Schwarz E M, Van Antwerp D, Miyamoto S (1995) Rel/NF-kappa B/I kappa B family: intimate tales of association and dissociation. Genes Dev 9:2723-273 5.
von Bulow V, Dubben S. Engelhardt G. Hebei S, Plumakers B, Heine H, Rink L, Haase H (2007) Zinc-dependent suppression of TNFalpha production is mediated by protein kinase A-induced inhibition of Raf-1I kappa B kinase beta, and NF-kappa B. J Immunol 179:4180-4186.
Ward et al., Nature 341:544-546, 1989
Wilhelm et al. (2004) Cancer Research, 64:7099-7109.
Wood et al., Nature 314:446-449, 1985
Yagami T (2006) Cerebral arachidonate cascade in dementia: Alzheimer's disease and vascular dementia. Curr Neuropharmacol 4:87-100.
Zhao L, Ma Q L, Calon F, Harris-White M E, Yang F, Lim G P, Morihara T et al. (2006) Role of p21-activated kinase pathway defects in the cognitive deficits of Alzheimer disease. Nat Neurosci 9:234-242.

1. A method for a) preventing and/or treating a neurodegenerative condition associated with Abeta peptide accumulation and/or aggregation in neural tissue in a human or animal, or for b) preventing or treating Alzheimer's disease-like neuropathology in a human or animal having trisomy 21 (Down's syndrome), said method comprising administering a therapeutically effective amount of a compound, or a composition comprising the compound, that inhibits function, activity, and/or expression of a Raf protein to a human or animal in need of treatment.

2. (canceled)

3. A method for a) preventing or inhibiting neuronal cell death and/or improving cell viability, or for b) decreasing the synthesis of Abeta peptide in a cell and/or decreasing oligomerization of Abeta peptide, or for c) inhibiting the activity and/or decreasing the expression of a Raf protein in a cell, or for d) decreasing or downregulating the expression of an inhibitor of NFκB in a cell, said method comprising contacting a cell with an effective amount of a compound, or a composition comprising the compound, that inhibits function or activity or expression of a Raf protein.

4-6. (canceled)

7. The method according to claim 1, wherein the Raf protein is Raf-1.

8. The method according to claim 1, wherein the Raf protein is a human Raf protein.

9. The method according to claim 1, wherein the compound is GW5074 having the structure: 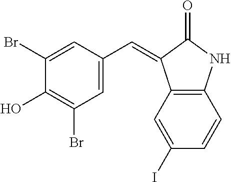
or an isomer or analog thereof, or a pharmaceutically acceptable salt thereof.

10. The method according to claim 1, wherein the compound is Sorafenib having the structure: 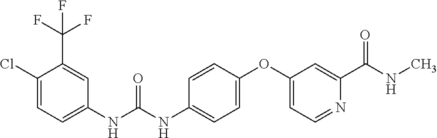
or an isomer or analog thereof, or a pharmaceutically acceptable salt thereof.

11. The method according to claim 1, wherein the compound is ZM336372.

12. The method according to claim 1, wherein the compound is an antibody or aptamer that binds to and inhibits activity and/or function of a Raf protein.

13. The method according to claim 1, wherein the compound is an antisense nucleic acid or interfering RNA or an siRNA that inhibits or interferes with expression of a Raf protein.

14. The method according to claim 3, wherein the cell is a cortical cell.

15. The method according to claim 1, wherein the neurodegenerative condition is Alzheimer's disease or Parkinson's disease.

16. (canceled)

17. The method according to claim 3, wherein the cell is a human cell.

18. A kit comprising in one or more containers:
i) a compound or composition that inhibits function, activity, and/or expression of a Raf protein; and optionally
ii) one or more compounds or compositions for treating a neurodegenerative disorder.

19. The kit according to claim 18, wherein said kit comprises sorafenib and/or GW5074 and/or ZM336372 and/or IκB-α, or an isomer or analog thereof, or a pharmaceutically acceptable salt thereof.

20. The kit according to claim 18, further comprising instructions and/or packaging materials that describe how to use and/or administer a compound or composition of the kit for treating a neurodegenerative disorder.

21. The kit according to claim 18, further comprising a pharmaceutically acceptable carrier and/or diluent.

22. The kit according to claim 18, wherein said kit comprises an antibody or aptamer that binds to and inhibits a Raf protein.

23. The kit according to claim 18, wherein said kit comprises an antisense nucleic acid and/or an interfering RNA and/or an siRNA and/or an miRNA that inhibit or interfere with expression of a Raf protein.

24. The method according to claim 3, wherein the Raf protein is Raf-1.

25. The method according to claim 3, wherein the Raf protein is a human Raf protein.

26. The method according to claim 3, wherein the compound is GW5074 having the structure: 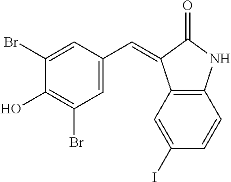
or an isomer or analog thereof, or a pharmaceutically acceptable salt thereof.

27. The method according to claim 3, wherein the compound is Sorafenib having the structure: 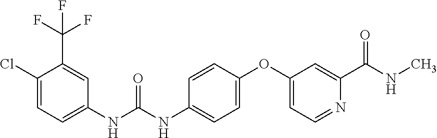
or an isomer or analog thereof, or a pharmaceutically acceptable salt thereof.

28. The method according to claim 3, wherein the compound is ZM336372.

29. The method according to claim 3, wherein the compound is an antibody or aptamer that binds to and inhibits activity and/or function of a Raf protein.

30. The method according to claim 3, wherein the compound is an antisense nucleic acid or interfering RNA or an siRNA that inhibits or interferes with expression of a Raf protein.
